(19) United States
(12) Patent Application Publication
(10) Pub. No.: US 2006/0106042 A1
(43) Pub. Date: May 18, 2006

(54) Substituted heterocyclpyrmidines

(76) Inventors: Rudiger Fischer, Pulheim (DE); Bernd Alig, Konigswinter (DE); Thomas Bretschneider, Lohmar (DE); Fritz Maurer, Lauffen (DE); Christoph Erdelen, Leichlingen (DE); Angelika Lubos-Erdelen, Leichlingen (DE); Jorg Konze, Koln (DE)

Correspondence Address: BAYER CROPSCIENCE LP;Patent Department, 100 BAYER ROAD, PITTSBURGH, PA, 15205-9741, US

(21) Appl. No.: 10/524,438

(22) PCT Filed: Aug. 11, 2003
(86) PCT No.: PCT/EP03/08881

§ 371 Date: Jun. 27, 2005

(30) Foreign Application Priority Data
Aug. 23, 2002 (DE) 10238725.7

(51) Int. Cl. A01N 43/54; C07D 417/02; C07D 413/02; C07D 403/02

(52) U.S. Cl. 514/269; 544/317

(57) ABSTRACT

The invention relates to substituted heterocyclylpyrimidines of formula (I) 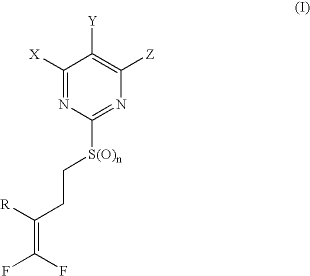 in which n is 0, 1, or 2, R is hydrogen of fluorine, and X, Y, and Z are as defined in the disclosure, to a method for their production, and to their use as pesticides.

The present invention relates to novel substituted heterocyclylpyrimidines, to various processes for their preparation and to their use as pesticides.

It is already known that certain substituted pyrimidines are suitable for controlling animal pests (cf. EP 0 506 270 A1/U.S. Pat. No. 5,246,938 A, WO 94/06777 A1/U.S. Pat. No. 5,684,011 A, WO 99/52 874 A1). However, these known substituted pyrimidines have, owing to various disadvantages, not attained any importance as pesticides.

This invention now provides novel substituted heterocyclylpyrimidines of the formula (I) 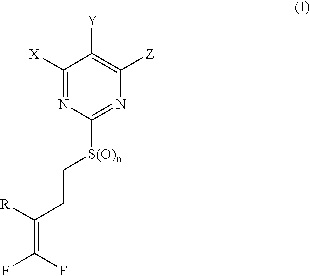
in which
n represents the numbers 0, 1 or 2,
R represents hydrogen or fluorine,
X, Y and Z independently of one another
represent hydrogen, nitro, cyano, carboxyl, carbamoyl, thiocarbamoyl, hydroxyl, cyanato, thiocyanato, halogen,
represent in each case optionally cyano-, halogen- or C₁-C₆-alkoxy-substituted alkyl, alkylcarbonyl, alkoxy, alkoxycarbonyl, alkylthio, alkylsulfinyl, alkylsulfonyl, alkylsulfonyloxy, alkylamino, alkylaminocarbonyl, alkoxycarbonylamino, alkylsulfonylamino, dialkylamino, dialkylaminocarbonyl or dialkylaminosulfonyl having in each case 1 to 6 carbon atoms in the alkyl groups,
represent in each case optionally halogen-substituted alkenyl, alkenyloxy, alkenyloxycarbonyl, alkynyl, alkynyloxy or alkynyloxycarbonyl having in each case 2 to 6 carbon atoms in the alkenyl or alkynyl groups,
represent cycloalkyl, cycloalkenyl or cycloalkylalkyl having in each case 3 to 6 carbon atoms in the cycloalkyl or cycloalkenyl group and, if appropriate, 1 to 4 carbon atoms in the alkyl moiety,
represent phenyl, phenoxy, phenylthio, pyridyl, furyl or thienyl, which are in each case optionally substituted by nitro, cyano, carboxyl, carbamoyl, thiocarbamoyl, halogen, C₁-C₄-alkyl, C₁-C₄-haloalkyl, C₁-C₄-alkoxy, C₁-C₄-haloalkoxy, C₁-C₄-alkylthio, C₁-C₄-haloalkylthio or C₁-C₄-alkoxy-carbonyl, or
together represent a benzo grouping or represent alkylene or alkenylene having in each case up to 4 carbon atoms, where the carbon chain may be interrupted by 1 to 3 nitrogen atoms or 1 to 2 (not directly adjacent) oxygen atoms and the ring thus formed is for its part optionally substituted by halogen or alkyl having 1 to 4 carbon atoms,
and at least one of the radicals X, Y or Z
represents a saturated or unsaturated monocyclic or bicyclic heterocyclyl grouping having up to 10 carbon atoms and up to 5 nitrogen atoms and/or one oxygen or sulfur atom which optionally additionally contains one or two oxo groupings (C═O), a thioxo grouping (C═S), a —SO grouping or a —SO₂ grouping and which optionally contains up to 4 substituents from the following list:
nitro; amino; cyano; carboxyl; carbamoyl; thiocarbamoyl; hydroxyl; halogen; in each case optionally cyano-, halogen- or C₁-C₆-alkoxy-substituted alkyl, alkylcarbonyl, alkoxy, alkoxycarbonyl, alkylthio, alkylsulfinyl, alkylsulfonyl, alkylsulfonyloxy, alkylamino, alkylaminocarbonyl, alkoxycarbonyl amino, alkylsulfonyl amino, dialkylamino, dialkylaminocarbonyl or dialkylaminosulfonyl having in each case 1 to 6 carbon atoms in the alkyl groups; in each case optionally halogen-substituted alkenyl, alkenyloxy, alkenyloxycarbonyl, alkynyl, alkynyloxy or alkynyloxycarbonyl having in each case 2 to 6 carbon atoms in the alkenyl or alkynyl groups; cycloalkyl, cycloalkenyl or cycloalkylalkyl having in each case 3 to 6 carbon atoms in the cycloalkyl or cycloalkenyl group and, if appropriate, 1 to 4 carbon atoms in the alkyl moiety; phenyl, benzyl, phenoxy, phenylthio, pyridyl, furyl or thienyl, which are in each case optionally substituted by nitro, cyano, carboxyl, carbamoyl, thiocarbamoyl, halogen, C₁-C₄-alkyl, C₁-C₄-haloalkyl, C₁-C₄-alkoxy, C₁-C₄-haloalkoxy, C₁-C₄-alkylthio, C₁-C₄-haloalkylthio or C₁-C₄-alkoxy-carbonyl.

Depending on the type and number of substituents, the compounds of the general formula (I) can, if appropriate, be present as geometrical and/or optical isomers or regioisomers or corresponding isomer mixtures of varying composition. What is claimed by the invention are both the pure isomers and the isomer mixtures. Likewise, depending on the type and number of substituents, compounds of the formula (I) can, if appropriate, be present in different tautomeric forms. The tautomers also form part of the subject-matter of the present invention.

The present invention furthermore provides all possible N-oxides which can be formed by compounds of the formula (I), and all salts of compounds of the formula (I), for example with mineral acids, such as hydrochloric acid.

Furthermore, it has been found that the novel substituted heterocyclylpyrimidines of the formula (I) are obtained when
(a) halogen-substituted pyrimidines of the formula (II), 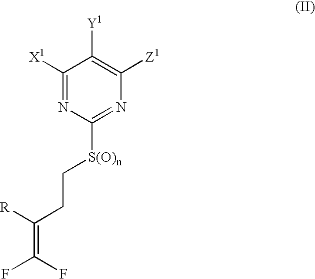
in which
n and R have the meanings given above,
X¹, Y¹ and Z¹ have in each case the meanings given above for the corresponding radicals X, Y and Z, but do not in any case represent a heterocyclyl grouping,
and at least one of the radicals X¹, Y¹ or Z¹ represents halogen, are reacted with heterocycles of the formula (IIIa), (IIIb) or (IIIc)
   H—X² (IIIa),
   H—Y² (IIIb),
   H-Z² (IIIc)
in which
X², Y² and Z² represent a saturated or unsaturated, monocyclic or bicyclic heterocyclyl grouping having up to 10 carbon atoms and up to 5 nitrogen atoms and/or one oxygen or sulfur atom which optionally additionally contains an oxo grouping (C═O), a thioxo grouping (C═S), a —SO grouping or a —SO₂ grouping and which optionally contains up to 4 substituents from the following list:
   nitro; amino; cyano; carboxyl; carbamoyl; thiocarbamoyl; hydroxyl; halogen; in each case optionally cyano-, halogen- or C₁-C₆-alkoxy-substituted alkyl, alkylcarbonyl, alkoxy, alkoxycarbonyl, alkylthio, alkylsulfinyl, alkylsulfonyl, alkylsulfonyloxy, alkylamino, alkylaminocarbonyl, alkoxycarbonylamino, alkylsulfonylamino, dialkylamino, dialkylaminocarbonyl or dialkylaminosulfonyl having in each case 1 to 6 carbon atoms in the alkyl groups; in each case optionally halogen-substituted alkenyl, alkenyloxy, alkenyloxycarbonyl, alkynyl, alkynyloxy or alkynyloxycarbonyl having in each case 2 to 6 carbon atoms in the alkenyl or alkynyl groups; cycloalkyl, cycloalkenyl or cycloalkylalkyl having in each case 3 to 6 carbon atoms in the cycloalkyl or cycloalkenyl group and, if appropriate, 1 to 4 carbon atoms in the alkyl moiety, phenyl, benzyl, phenoxy, phenylthio, pyridyl, furyl or thienyl, which are in each case optionally substituted by nitro, cyano, carboxyl, carbamoyl, thiocarbamoyl, halogen, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₂-alkylenedioxy or C₁-C₂-haloalkylenedioxy, or C₁-C₄-alkoxy-carbonyl,
      if appropriate in the presence of one or more reaction auxiliaries and if appropriate in the presence of one or more diluents,
      or when
(b) heterocyclylpyrimidinethiols of the formula (IV), 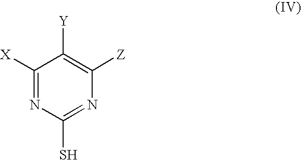
in which
X, Y and Z have the meanings given above,
are reacted with fluoroalkenyl halides of the formula (V), 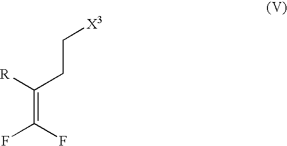
in which
R has the meanings given above and
x³ represents halogen,
if appropriate in the presence of one or more reaction auxiliaries and if appropriate in the presence of one or more diluents,
and, if appropriate, the compounds of the formula (I) obtained according to process (a) or (b) are in a customary manner converted into other compounds of the formula (I) according to the above definition.

Finally, it has been found that the novel substituted heterocyclylpyrimidines of the formula (I) are highly effective against animal pests, in particular against insects, arachnids and nematodes, especially against nematodes, and can be used both in crop protection and in the protection of materials for controlling animal pests.

In the definitions, the hydrocarbon chains, such as alkyl or alkenyl, are in each case straight-chain or branched—including in combination with heteroatoms, such as in alkoxy.

Optionally substituted radicals can be mono- or polysubstituted, where in the case of polysubstitution, the substituents can be identical or different.

Preferred substituents or ranges of the radicals present in the formulae given above and below are listed below.

n preferably represents the numbers 0 or 2.

R preferably represents hydrogen.

R furthermore preferably represents fluorine.
X, Y and Z independently of one another
   represent hydrogen, nitro, cyano, carboxyl, carbamoyl, thiocarbamoyl, hydroxyl, cyanato, thiocyanato, halogen,
   represent in each case optionally cyano-, fluorine-, chlorine-, bromine- or C₁-C₄-alkoxy-substituted alkyl, alkylcarbonyl, alkoxy, alkoxycarbonyl, alkylthio, alkylsulfinyl, alkylsulfonyl, alkylsulfonyloxy, alkylamino, alkylaminocarbonyl, alkoxycarbonylamino, alkylsulfonylamino, dialkylamino, dialkylaminocarbonyl or dialkylaminosulfonyl having in each case 1 to 5 carbon atoms in the alkyl groups,
   represent in each case optionally fluorine-, chlorine- or bromine-substituted alkenyl, alkenyloxy, alkenyloxycarbonyl, alkynyl, alkynyloxy or alkynyloxycarbonyl having in each case 2 to 5 carbon atoms in the alkenyl or alkynyl groups,
   represent cycloalkyl, cycloalkenyl or cycloalkylalkyl having in each case 3 to 6 carbon atoms in the cycloalkyl or cycloalkenyl group and, if appropriate, 1 to 3 carbon atoms in the alkyl moiety, or
   represent phenyl, phenoxy, phenylthio, pyridyl, furyl or thienyl which are in each case optionally substituted by nitro, cyano, carboxyl, carbamoyl, thiocarbamoyl, halogen, by C₁-C₄-alkyl, C₁-C₄-haloalkyl, C₁-C₄-alkoxy, C₁-C₄-haloalkoxy, C₁-C₄-alkylthio, C₁-C₄-haloalkylthio (where in each case halogen preferably represents fluorine or chlorine) or C₁-C₄-alkoxy-carbonyl, or
   together represent a benzo grouping or represent alkylene or alkenylene having in each case up to 4 carbon atoms, where the carbon chain may be interrupted by 1 to 3 nitrogen atoms or 1 to 2 (not directly adjacent) oxygen atoms and the ring thus formed is for its part optionally substituted by fluorine, chlorine, bromine or alkyl having 1 to 3 carbon atoms, and where at least one of the radicals X, Y or Z represents a saturated or unsaturated, monocyclic or bicyclic heterocyclyl grouping having 4, 6, 9 or 10 carbon atoms and up to 4 nitrogen atoms and/or one oxygen or sulfur atom which optionally additionally contains one or two oxo groupings (C═O), a thioxo grouping (C═S), a —SO grouping or a —SO₂ grouping and which optionally contains up to 3 substituents from the following list:
   nitro; amino; cyano; carboxyl; carbamoyl; thiocarbamoyl; hydroxyl; halogen; in each case optionally cyano-, fluorine-, chlorine-, bromine- or C₁-C₄-alkoxy-substituted alkyl, alkylcarbonyl, alkoxy, alkoxycarbonyl, alkylthio, alkylsulfinyl, alkylsulfonyl, alkylsulfonyloxy, alkylamino, alkylaminocarbonyl, alkoxycarbonylamino, alkylsulfonylamino, dialkylamino, dialkylaminocarbonyl or dialkylaminosulfonyl having in each case 1 to 5 carbon atoms in the alkyl groups; in each case optionally halogen-substituted alkenyl, alkenyloxy, alkenyloxycarbonyl, alkynyl, alkynyloxy or alkynyloxycarbonyl having in each case 2 to 5 carbon atoms in the alkenyl or alkynyl groups; cycloalkyl, cycloalkenyl or cycloalkylalkyl having in each case 3 to 6 carbon atoms in the cycloalkyl or cycloalkenyl group and, if appropriate, 1 to 3 carbon atoms in the alkyl moiety, phenyl, benzyl, phenoxy, phenylthio, pyridyl, furyl or thienyl, which are in each case optionally substituted by nitro, cyano, carboxyl, carbamoyl, thiocarbamoyl, halogen, by C₁-C₄-alkyl, C₁-C₄-haloalkyl, C₁-C₄-alkoxy, C₁-C₄-haloalkoxy, C₁-C₄-alkylthio, C₁-C₄-haloalkylthio (where in each case halogen preferably represents fluorine or chlorine), C₁-C₄-alkoxy-carbonyl, methylenedioxy or difluoromethylenedioxy.

n particularly preferably represents 0.
X, Y and Z independently of one another
   particularly preferably represent hydrogen, nitro, cyano, carboxyl, carbamoyl, thiocarbamoyl, hydroxyl, cyanato, thiocyanato, fluorine, chlorine, bromine,
   particularly preferably represent in each case optionally cyano-, fluorine-, chlorine-, bromine-, methoxy-, ethoxy-, n- or i-propoxy-substituted methyl, ethyl, n- or i-propyl, n-, i-, s- or t-butyl, acetyl, propionyl, n- or i-butyroyl, methoxy, ethoxy, n- or i-propoxy, n-, i-, s- or t-butoxy, methoxycarbonyl, ethoxycarbonyl, n- or i-propoxycarbonyl, n-, i-, s- or t-butoxycarbonyl, methylthio, ethylthio, n- or i-propylthio, n-, i-, s- or t-butylthio, methylsulfinyl, ethylsulfinyl, n- or i-propylsulfinyl, methylsulfonyl, ethylsulfonyl, n- or i-propylsulfonyl, methylsulfonyloxy, ethylsulfonyloxy, n- or i-propylsulfonyloxy, methylamino, ethylamino, n- or i-propylamino, n-, i-, s- or t-butylamino, methylaminocarbonyl, ethylaminocarbonyl, n- or i-propylaminocarbonyl, n-, i-, s- or t-butylaminocarbonyl, methoxycarbonylamino, ethoxycarbonylamino, n- or i-propoxycarbonylamino, n-, i-, s- or t-butoxycarbonylamino, methylsulfonylamino, ethylsulfonylamino, n- or i-propylsulfonylamino, n-, i-, s- or t-butylsulfonylamino, dimethylamino, diethylamino, di-n-propyl-amino, di-i-propylamino, dimethylaminocarbonyl, diethylaminocarbonyl, dimethylaminosulfonyl or diethylaminosulfonyl,
   particularly preferably represent in each case optionally fluorine-, chlorine- or bromine-substituted ethenyl, propenyl, butenyl, propenyloxy, butenyloxy, propenyloxycarbonyl, butenyloxycarbonyl, ethynyl, propynyl, butynyl, propynyloxy, butynyloxy, propynyloxycarbonyl or butynyloxycarbonyl,
   particularly preferably represent cyclopropyl, cyclobutyl, cyclopentyl, cyclohexyl, cyclopentenyl, cyclohexenyl, cyclopropylmethyl, cyclobutylmethyl, cyclopentylmethyl or cyclohexylmethyl, or
   particularly preferably represent phenyl, phenoxy, phenylthio, pyridyl, furyl or thienyl, which are in each case optionally substituted by nitro, cyano, carboxyl, carbamoyl, thiocarbamoyl, fluorine, chlorine, bromine, by methyl, ethyl, n- or i-propyl, n-, i-, s- or t-butyl, difluoromethyl, dichloromethyl, trifluoromethyl, trichloromethyl, chlorodifluoromethyl, fluorodichloromethyl, methoxy, ethoxy, n- or i-propoxy, n-, i-, s- or t-butoxy, difluoromethoxy, trifluoromethoxy, chlorodifluoromethoxy, methylthio, ethylthio, n- or i-propylthio, n-, i-, s- or t-butylthio, difluoromethylthio, trifluoromethylthio, chlorodifluoromethylthio, methoxycarbonyl, ethoxycarbonyl, n- or i-propoxycarbonyl, n-, i-, s- or t-butoxycarbonyl, or
   together particularly preferably represent trimethylene, tetramethylene, propene-1,3-diyl or butadiene1,4-diyl, where the carbon chain may be interrupted by 1 to 3 nitrogen atoms or 1 to 2 (not directly adjacent) oxygen atoms and the ring thus formed is for its part optionally substituted by fluorine, chlorine, bromine, methyl or ethyl, and where
   at least one of the radicals X, Y or Z particularly preferably represents a saturated or unsaturated, monocyclic or bicyclic heterocyclyl grouping from the group consisting of furyl, benzofuryl, tetrahydrofuryl, thienyl, benzothienyl, pyrrolyl, indazolyl, tetrahydroindazolyl, oxopyrrolyl, pyrrolinyl, pyrrolidinyl, 2,5-dioxo-1-azacyclopentyl, pyrazolyl, pyrazolinyl, oxopyrazolinyl, 2-oxo-1,3-diazacyclopentyl, imidazolyl, triazolyl, benzotriazolyl, oxotriazolinyl, tetrazolyl, oxazolyl, benzoxazolyl, thiazolyl, benzothiazolyl, oxadiazolyl, isoxazolyl, thiadiazolyl, pyridinyl, 1,4-dihydro-4-oxopyridin-1-yl, quinolinyl, isoquinolinyl, piperidinyl, oxopiperidinyl, pyrazinyl, pyridazinyl, pyrimidinyl, 2-oxo-1,3-diazacyclohexyl, morpholinyl, which optionally contains up to 3 substituents from the following list:
   nitro; amino; cyano; carboxyl; carbamoyl; thiocarbamoyl; hydroxyl; fluorine; chlorine; bromine; iodine; in each case optionally cyano-, fluorine-, chlorine-, bromine-, methoxy-, ethoxy-, n- or i-propoxy-substituted methyl, ethyl, n- or i-propyl, n-, i-, s- or t-butyl, acetyl, propionyl, n- or i-butyroyl, methoxy, ethoxy, n- or i-propoxy, n-, i-, s- or t-butoxy, methoxycarbonyl, ethoxycarbonyl, n- or i-propoxycarbonyl, n-, i-, s- or t-butoxycarbonyl, methylthio, ethylthio, n- or i-propylthio, n-, i-, s- or t-butylthio, methylsulfinyl, ethylsulfinyl, n- or i-propylsulfinyl, methylsulfonyl, ethylsulfonyl, n- or i-propylsulfonyl, methylsulfonyloxy, ethylsulfonyloxy, n- or i-propylsulfonyloxy, methylamino, ethylamino, n- or i-propylamino, n-, i-, s- or t-butylamino, methylaminocarbonyl, ethylaminocarbonyl, n- or i-propylaminocarbonyl, n-, i-, s- or t-butylaminocarbonyl, methoxycarbonylamino, ethoxycarbonylamino, n- or i-propoxycarbonylamino, n-, i-, s- or t-butoxycarbonylamino, methylsulfonylamino, ethylsulfonylamino, n- or i-propylsulfonylamino, n-, i-, s- or t-butylsulfonylamino, dimethylamino, diethylamino, di-n-propylamino, di-i-propylamino, dimethylaminocarbonyl, diethylaminocarbonyl, dimethylaminosulfonyl or diethylaminosulfonyl; in each case optionally fluorine-, chlorine- or bromine-substituted ethenyl, propenyl, butenyl, propenyloxy, butenyloxy, propenyloxycarbonyl, butenyloxycarbonyl, ethynyl, propynyl, butynyl, propynyloxy, butynyloxy, propynyloxycarbonyl or butynyloxycarbonyl, cyclopropyl, cyclobutyl, cyclopentyl, cyclohexyl, cyclopentenyl, cyclohexenyl, cyclopropylmethyl, cyclobutylmethyl, cyclopentylmethyl or cyclohexylmethyl, phenyl, benzyl, phenoxy, phenylthio, pyridyl, furyl or thienyl, which are in each case optionally substituted by nitro, cyano, carboxyl, carbamoyl, thiocarbamoyl, fluorine, chlorine, bromine, by methyl, ethyl, n- or i-propyl, n-, i-, s- or t-butyl, difluoromethyl, dichloromethyl, trifluoromethyl, trichloromethyl, chlorodifluoromethyl, fluorodichloromethyl, methoxy, ethoxy, n- or i-propoxy, n-, i-, s- or t-butoxy, difluoromethoxy, trifluoromethoxy, chlorodifluoromethoxy, methylthio, ethylthio, n- or i-propylthio, n-, i-, s- or t-butylthio, difluoromethylthio, trifluoromethylthio, chlorodifluoromethylthio, methoxycarbonyl, ethoxycarbonyl, n- or i-propoxycarbonyl, n-, i-, s- or t-butoxycarbonyl, methylenedioxy or difluoromethylenedioxy.
X, Y and Z independently of one another very particularly preferably represent hydrogen or represent a saturated or unsaturated, monocyclic or bicyclic heterocyclyl grouping from the group consisting of thienyl, benzothienyl, pyrrolyl, pyrazolyl, triazolyl, thiazolyl, benzothiazolyl, pyridinyl, quinolinyl, pyridazinyl or morpholinyl, which optionally contains up to 2, preferably 1, substituents from the following list:
   nitro; amino; cyano; carbamoyl; thiocarbamoyl; fluorine; chlorine; bromine; iodine; in each case optionally fluorine-, chlorine-, methoxy- or ethoxy-substituted methyl, ethyl, n- or i-propyl, methoxy, ethoxy, n- or i-propoxy, methoxycarbonyl, ethoxycarbonyl, n- or i-propoxycarbonyl, methylthio, ethylthio, n- or i-propylthio, methylsulfonyl, ethylsulfonyl, n- or i-propylsulfonyl; in each case optionally fluorine- or chlorine-substitutes phenyl,
   where Y preferably represents hydrogen and one of the two radicals X or Z represents one of the heterocycles mentioned, while the remaining of the two radicals X or Z again represents hydrogen.

A very particularly preferred group are those compounds of the formula (I) in which
n, R, X, Y and Z have the meanings given above as particularly preferred, where Y preferably represents hydrogen,
   and at least one of the radicals
X or Z represents a saturated or unsaturated, monocyclic or bicyclic heterocyclyl grouping from the group consisting of furyl, benzofuryl, tetrahydrofuryl, thienyl, benzothienyl, pyrrolyl, indazolyl, tetrahydroindazolyl, oxopyrrolyl, pyrrolinyl, pyrrolidinyl, 2,5-dioxo-1-azacyclopentyl, pyrazolyl, pyrazolinyl, oxopyrazolinyl, 2-oxo-1,3-diazacyclopentyl, imidazolyl, triazolyl, benzotriazolyl, oxotriazolinyl, tetrazolyl, oxazolyl, benzoxazolyl, thiazolyl, benzothiazolyl, oxadiazolyl, isoxazolyl, thiadiazolyl, pyridinyl, 1,4-dihydro-4-oxopyridin-1-yl, quinolinyl, isoquinolinyl, piperidinyl, oxopiperidinyl, pyrazinyl, pyridazinyl, pyrimidinyl, 2-oxo-1,3-diazacyclohexyl, morpholinyl, which optionally contains up to 3 substituents from the following list:
   nitro; cyano; carboxyl; carbamoyl; thiocarbamoyl; hydroxyl; fluorine; chlorine; bromine; iodine; in each case optionally cyano-, fluorine-, chlorine-, bromine-, methoxy-, ethoxy-, n- or i-propoxy-substituted methyl, ethyl, n- or i-propyl, n-, i-, s- or t-butyl, acetyl, propionyl, n- or i-butyroyl, methoxy, ethoxy, n- or i-propoxy, n-, i-, s- or t-butoxy, methoxycarbonyl, ethoxycarbonyl, n- or i-propoxycarbonyl, n-, i-, s- or t-butoxycarbonyl, methylthio, ethylthio, n- or i-propylthio, n-, i-, s- or t-butylthio, methylsulfinyl, ethylsulfinyl, n- or i-propylsulfinyl, methylsulfonyl, ethylsulfonyl, n- or i-propylsulfonyl, methylsulfonyloxy, ethylsulfonyloxy, n- or i-propylsulfonyloxy, methylamino, ethylamino, n- or i-propylamino, n-, i-, s- or t-butylamino, methylaminocarbonyl, ethylaminocarbonyl, n- or i-propylaminocarbonyl, n-, i-, s- or t-butylaminocarbonyl, methoxycarbonylamino, ethoxycarbonylamino, n- or i-propoxycarbonylamino, n-, i-, s- or t-butoxycarbonylamino, methylsulfonylamino, ethylsulfonylamino, n- or i-propylsulfonylamino, n-, i-, s- or t-butylsulfonylamino, dimethylamino, diethylamino, di-n-propyl-amino, di-i-propylamino, dimethylaminocarbonyl, diethylaminocarbonyl, dimethylaminosulfonyl or diethylaminosulfonyl; in each case optionally fluorine-, chlorine- or bromine-substituted ethenyl, propenyl, butenyl, propenyloxy, butenyloxy, propenyloxycarbonyl, butenyloxycarbonyl, ethynyl, propynyl, butynyl, propynyloxy, butynyloxy, propynyloxycarbonyl or butynyloxycarbonyl; cyclopropyl, cyclobutyl, cyclopentyl, cyclohexyl, cyclopentenyl, cyclohexenyl, cyclopropylmethyl, cyclobutylmethyl, cyclopentylmethyl or cyclohexylmethyl, phenyl, benzyl, phenoxy, phenylthio, pyridyl, furyl or thienyl, which are in each case optionally substituted by nitro, cyano, carboxyl, carbamoyl, thiocarbamoyl, fluorine, chlorine, bromine, by methyl, ethyl, n- or i-propyl, n-, i-, s- or t-butyl, difluoromethyl, dichloromethyl, trifluoromethyl, trichloromethyl, chlorodifluoromethyl, fluorodichloromethyl, methoxy, ethoxy, n- or i-propoxy, n-, i-, s- or t-butoxy, difluoromethoxy, trifluoromethoxy, chlorodifluoromethoxy, methylthio, ethylthio, n- or i-propylthio, n-, i-, s- or t-butylthio, difluoromethylthio, trifluoromethylthio, chlorodifluoromethylthio, methoxycarbonyl, ethoxycarbonyl, n- or i-propoxycarbonyl, n-, i-, s- or t-butoxycarbonyl, methylenedioxy or difluoromethylenedioxy,
   and where the optionally remaining radical X or Z represents hydrogen.

A further very particularly preferred group are those compounds of the formula (I) in which
n, R, X and Z have the meanings given above as particularly preferred, where X and Z preferably represent hydrogen, and
Y represents a saturated or unsaturated, monocyclic or bicyclic heterocyclyl grouping from the group consisting of furyl, benzofuryl, tetrahydrofuryl, thienyl, benzothienyl, pyrrolyl, oxopyrrolyl, pyrrolinyl, pyrrolidinyl, pyrazolyl, pyrazolinyl, oxopyrazolinyl, 2-oxo-1,3-diazacyclopentyl, triazolyl, benzotriazolyl, oxotriazolinyl, tetrazolyl, oxazolyl, benzoxazolyl, thiazolyl, benzothiazolyl, oxadiazolyl, isoxazolyl, thiadiazolyl, pyridinyl, quinolinyl, isoquinolinyl, piperidinyl, oxopiperidinyl, pyrazinyl, pyridazinyl, pyrimidinyl, 2-oxo-1,3-diazacyclohexyl, morpholinyl, which optionally contains up to 3 substituents from the following list:
   nitro; cyano; carboxyl; carbamoyl; thiocarbamoyl; hydroxyl; fluorine; chlorine; bromine; iodine; in each case optionally cyano-, fluorine-, chlorine-, bromine-, methoxy-, ethoxy-, n- or i-propoxy-substituted methyl, ethyl, n- or i-propyl, n-, i-, s- or t-butyl, acetyl, propionyl, n- or i-butyroyl, methoxy, ethoxy, n- or i-propoxy, n-, i-, s- or t-butoxy, methoxycarbonyl, ethoxycarbonyl, n- or i-propoxycarbonyl, n-, i-, s- or t-butoxycarbonyl, methylthio, ethylthio, n- or i-propylthio, n-, i-, s- or t-butylthio, methylsulfinyl, ethylsulfinyl, n- or i-propylsulfinyl, methylsulfonyl, ethylsulfonyl, n- or i-propylsulfonyl, methylsulfonyloxy, ethylsulfonyloxy, n- or i-propylsulfonyloxy, methylamino, ethylamino, n- or i-propylamino, n-, i-, s- or t-butylamino, methylaminocarbonyl, ethylaminocarbonyl, n- or i-propylaminocarbonyl, n-, i-, s- or t-butylaminocarbonyl, methoxycarbonylamino, ethoxycarbonylamino, n- or i-propoxycarbonylamino, n-, i-, s- or t-butoxycarbonylamino, methylsulfonylamino, ethylsulfonylamino, n- or i-propylsulfonylamino, n-, i-, s- or t-butylsulfonylamino, dimethylamino, diethylamino, di-n-propyl-amino, di-i-propylamino, dimethylaminocarbonyl, diethylaminocarbonyl, dimethylaminosulfonyl or diethylaminosulfonyl; in each case optionally fluorine-, chlorine- or bromine-substituted ethenyl, propenyl, butenyl, propenyloxy, butenyloxy, propenyloxycarbonyl, butenyloxycarbonyl, ethynyl, propynyl, butynyl, propynyloxy, butynyloxy, propynyloxycarbonyl or butynyloxycarbonyl; cyclopropyl, cyclobutyl, cyclopentyl, cyclohexyl, cyclopentenyl, cyclohexenyl, cyclopropylmethyl, cyclobutylmethyl, cyclopentylmethyl or cyclohexylmethyl, phenyl, benzyl, phenoxy, phenylthio, pyridyl, furyl or thienyl, which are in each case optionally substituted by nitro, cyano, carboxyl, carbamoyl, thiocarbamoyl, fluorine, chlorine, bromine, by methyl, ethyl, n- or i-propyl, n-, i-, s- or t-butyl, difluoromethyl, dichloromethyl, trifluoromethyl, trichloromethyl, chlorodifluoromethyl, fluorodichloromethyl, methoxy, ethoxy, n- or i-propoxy, n-, i-, s- or t-butoxy, difluoromethoxy, trifluoromethoxy, chlorodifluoromethoxy, methylthio, ethylthio, n- or i-propylthio, n-, i-, s- or t-butylthio, difluoromethylthio, trifluoromethylthio, chlorodifluoromethylthio, methoxycarbonyl, ethoxycarbonyl, n- or i-propoxycarbonyl, n-, i-, s- or t-butoxycarbonyl.

Compounds of the formula (I) in which R in each case represents hydrogen or fluorine are equally preferred. Both groups of compounds form, independently of one another, part of the subject-matter of the present invention.

Preference according to the invention is given to the compounds of the formula (I) which contain a combination of the meanings given above as being preferred.

Particular preference according to the invention is given to the compounds of the formula (I) which contain a combination of the meanings given above as being particularly preferred.

Very particular preference according to the invention is given to the compounds of the formula (I) which contain a combination of the meanings given above as being very particularly preferred.

However, the general or preferred radical definitions or illustrations given above can also be combined with one another as desired, i.e. including combinations between the respective ranges and preferred ranges. The definitions apply both to the end products and, correspondingly, to precursors and intermediates.

Using, for example, 4-chloro-6-methyl-2-[(3,4,4-trifluoro-3-butenyl)thio]pyrimidine and morpholine as starting materials, the course of the process (a) according to the invention can be illustrated by the formula scheme below:

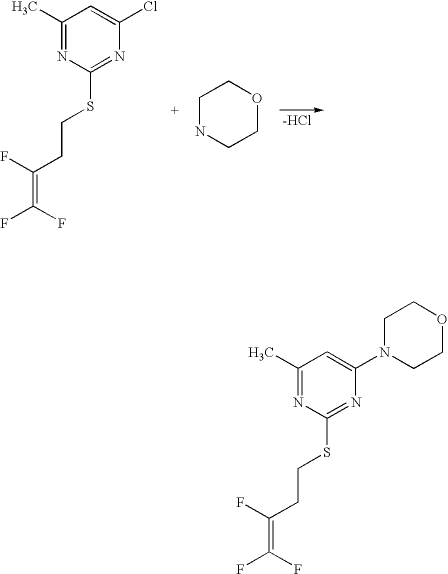

Using, for example, 4-(pyridin-2-yl)-pyrimidine-2-thiol and 4-bromo-1,1-difluoro-1-butene as starting materials, the course of the process (b) according to the invention can be illustrated by the formula scheme below:

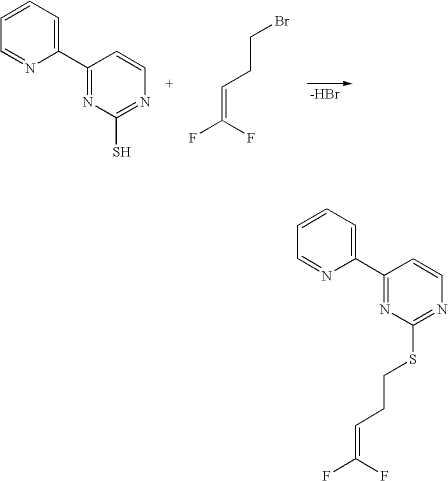

The formula (II) provides a general definition of the halogen-substituted pyrimidines to be used as starting materials for carrying out the process (a) according to the invention. In the formula (II), n and R preferably or particularly preferably have those meanings which have already been mentioned in connection witht the description of the compounds of the formula (I) according to the invention as being preferred, particularly preferred for n and R. X¹ preferably has the meaning given above for X as being preferred, particularly preferred or very particularly preferred; Y¹ preferably has the meaning given above for Y as being preferred, particularly preferred or very particularly preferred and Z¹ preferably has the meaning given above for Z as being preferred, particularly preferred or very particularly preferred, where at least one of the radicals X¹, Y¹ or Z¹ represents halogen, preferably fluorine, chlorine, bromine or iodine, in particular fluorine, chlorine or bromine, and none of these radicals represents heterocyclyl.

Most of the starting materials of the general formula (II) are known, and/or they can be prepared by processes known per se (cf., in particular, EP 506 270 A1 and WO 94/06 777 A1 and the Preparation Examples).

Thus, the halogen-substituted pyrimidines of the formula (II) are obtained when corresponding hydroxypyrimidines of the general formula (VI) 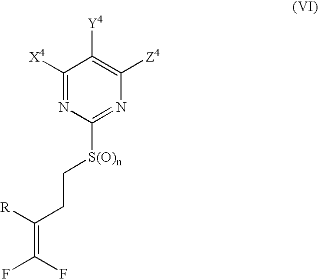
in which
n and R have the meaning given above,
X⁴, Y⁴ and Z⁴ have in each case the meanings given above for the corresponding radicals X, Y and Z, but do not in any case represent a heterocyclyl grouping,
and at least one of the radicals X⁴, Y⁴ or Z⁴ represents hydroxyl,
are reacted with a “halogenating agent”, i.e. a chemical suitable for introducing halogen substituents in place of hydroxyl groups into heterocycles, such as, for example, phosphoryl chloride (“phosphorus oxychloride”), thionyl chloride or phosgene, at temperatures between 0° C. and 150° C. (cf. the Preparation Examples).

The hydroxypyrimidines of the general formula (VI) required as precursors for this purpose are known, and/or they can be prepared by processes known per se (cf. EP 506 270 A1/U.S. Pat. No. 5,246,938 A, WO 94/06777 A1/U.S. Pat. No. 5,684,011 A, Preparation Examples).

The formulae (IIIa), (IIIb) and (IIIc) provide general definitions of the heterocycles further to be used as starting materials in the process (a) according to the invention for preparing compounds of the formula (I). In these formulae, X² and/or Y² and/or Z² preferably have that meaning which has been mentioned above as being preferred, particularly preferred or very particularly preferred for the heterocyclyl grouping.

The heterocycles of the formulae (IIIa), (IIIb) and (IIIc) are known organic chemicals.

The formula (IV) provides a general definition of the heterocyclylpyrimidinethiols to be used as starting materials for carrying out the process (b) according to the invention. In the formula (IV), X, Y and Z preferably have those meanings which have already been mentioned in connection with the description of the compounds of the formula (I) according to the invention as being preferred, particularly preferred or very particularly preferred for X, Y and Z.

The starting materials of the formula (IV) are known, and/or they can be prepared by processes known per se (cf. Dokl. Bolg. Akad. Nauk 24 (1971), 247-250—cited in Chem. Abstracts 75:5841; Aust. J. Chem. 33 (1980), 2291-2298; Organic Preparations and Procedures International 29 (1997), 285-292; WO 98/27092; Tetrahedron Lett. 40 (1999), 4779-4782).

The formula (V) provides a general definition of the fluoroalkenyl halides further to be used as starting materials in the process (b) according to the invention for preparing compounds of the formula (I). In the formula (V), R preferably has that meaning which has been mentioned above as being preferred or as being particularly preferred for R; X³ preferably represents fluorine, chlorine, bromine or iodine, in particular chlorine or bromine.

The starting materials of the formula (V) are known and/or can be prepared by known processes (cf. EP 334 796 A1, EP 660 827 A1, EP 712 395 A1, EP 749 433 A1, EP 850 211 A1).

The processes (a) and (b) according to the invention for preparing the compounds of the general formula (I) are preferably carried out using one or more reaction auxiliaries. Reaction auxiliaries suitable for the processes according to the invention are, in general, the customary inorganic or organic bases or acid acceptors. These preferably include alkali metal or alkaline earth metal acetates, amides, carbonates, bicarbonates, hydrides, hydroxides or alkoxides, such as, for example, sodium acetate, potassium acetate or calcium acetate, lithium amide, sodium amide, potassium amide or calcium amide, sodium carbonate, potassium carbonate, cesium carbonate or calcium carbonate, sodium bicarbonate, potassium bicarbonate or calcium bicarbonate, lithium hydride, sodium hydride, potassium hydride or calcium hydride, lithium hydroxide, sodium hydroxide, potassium hydroxide or calcium hydroxide, sodium methoxide, ethoxide, n- or i-propoxide, n-, i-, s- or t-butoxide or potassium methoxide, ethoxide, n- or i-propoxide, n-, i-, s- or t-butoxide; furthermore also basic organic nitrogen compounds, such as, for example, trimethylamine, triethylamine, tripropylamine, tributylamine, ethyl-diisopropylamine, N,N-dimethylcyclohexylamine, dicyclohexylamine, ethyl-dicyclohexylamine, N,N-dimethylaniline, N,N-dimethyl-benzylamine, pyridine, 2-methyl-, 3-methyl-, 4-methyl-, 2,4-dimethyl-, 2,6-dimethyl-, 3,4-dimethyl- and 3,5-dimethyl-pyridine, 5-ethyl-2-methylpyridine, 4-dimethylamino-pyridine, N-methyl-piperidine, 1,4-diazabicyclo[2,2,2]-octane (DABCO), 1,5-diazabicyclo[4,3,0]-non-5-ene (DBN), or 1,8-diazabicyclo-[5,4,0]-undec-7-ene (DBU).

The processes (a) and (b) according to the invention for preparing the compounds of the general formula (I) are preferably carried out using one or more diluents. Diluents suitable for carrying out the processes (a) and (b) according to the invention are especially inert organic solvents. These include, in particular, aliphatic, alicyclic or aromatic, optionally halogenated hydrocarbons, such as, for example, benzine, benzene, toluene, xylene, chlorobenzene, dichlorobenzene, petroleum ether, hexane, cyclohexane, dichloromethane, chloroform, carbon tetrachloride; ethers, such as diethyl ether, diisopropyl ether, dioxane, tetrahydrofuran or ethylene glycol dimethyl ether or ethylene glycol diethyl ether; ketones, such as acetone, butanone or methyl isobutyl ketone; nitriles, such as acetonitrile, propionitrile or butyronitrile; amides, such as N,N-dimethylformamide, N,N-dimethylacetamide, N-methyl-formanilide, N-methyl-pyrrolidone or hexamethylphosphoric triamide; esters such as methyl acetate or ethyl acetate, sulfoxides, such as dimethyl sulfoxide, alcohols, such as methanol, ethanol, n- or i-propanol, ethylene glycol monomethyl ether, ethylene glycol monoethyl ether, diethylene glycol monomethyl ether, diethylene glycol monoethyl ether, mixtures thereof with water or pure water.

When carrying out the processes (a) and (b) according to the invention, the reaction temperatures can be varied within a relatively wide range. In general, the processes are carried out at temperatueres between 0° C. and 150° C., preferably between 10° C. and 120° C.

The processes (a) and (b) according to the invention are generally carried out under atmospheric pressure. However, it is also possible to carry out the processes according to the invention under elevated or reduced pressure—in general between 0.1 bar and 10 bar.

For carrying out the processes (a) and (b) according to the invention, the starting materials are generally employed in approximately equimolar amounts. However, it is also possible to use a relatively large excess of one of the components. The reaction is generally carried out in a suitable diluent in the presence of a reaction auxiliary, and the reaction mixture is generally stirred for a number of hours at the required temperature. Work-up is carried out by customary methods (cf. the Preparation Examples).

The active compounds are suitable for controlling animal pests, in particular insects, arachnids and nematodes, which are encountered in agriculture, in forests, in the protection of stored products and of materials, and in the hygiene sector, and have good plant tolerance and favorable toxicity to warm-blooded animals. They may preferably be employed as crop protection agents. They are active against normally sensitive and resistant species and against all or some stages of development. The abovementioned pests include:

From the order of the Isopoda, for example, Oniscus asellus, Armadillidium vulgare and Porcellio scaber.

From the order of the Diplopoda, for example, Blaniulus guttulatus.

From the order of the Chilopoda, for example, Geophilus carpophagus and Scutigera spp.

From the order of the Symphyla, for example, Scutigerella immaculata.

From the order of the Thysanura, for example, Lepisma saccharina.

From the order of the Collembola, for example, Onychiurus armatus.

From the order of the Orthoptera, for example, Acheta domesticus, Gryllotalpa spp., Locusta migratoria migratorioides, Melanoplus spp. and Schistocerca gregaria.

From the order of the Blattaria, for example, Blatta orientalis, Periplaneta americana, Leucophaea maderae, Blattella germanica.

From the order of the Dermaptera, for example, Forficula auricularia.

From the order of the Isoptera, for example, Reticulitermes spp.

From the order of the Phthiraptera, for example, Pediculus humanus corporis, Haematopinus spp., Linognathus spp., Trichodectes spp. and Damalinia spp.

From the order of the Thysanoptera, for example, Hercinothrips femoralis, Thrips tabaci, Thrips palmi and Frankliniella accidentalis.

From the order of the Heteroptera, for example, Eurygaster spp., Dysdercus intermedius, Piesma quadrata, Cimex lectularius, Rhodnius prolixus and Triatoma spp.

From the order of the Homoptera, for example, Aleurodes brassicae, Bemisia tabaci, Trialeurodes vaporariorum, Aphis gossypii, Brevicoryne brassicae, Cryptomyzus ribis, Aphis fabae, Aphis pomi, Eriosoma lanigerum, Hyalopterus arundinis, Phylloxera vastatrix, Pemphigus spp., Macrosiphum avenae, Myzus spp., Phorodon humuli, Rhopalosiphum padi, Empoasca spp., Euscelis bilobatus, Nephotettix cincticeps, Lecanium corni, Saissetia oleae, Laodelphax striatellus, Nilaparvata lugens, Aonidiella aurantii, Aspidiotus hederae, Pseudococcus spp. and Psylla spp.

From the order of the Lepidoptera, for example, Pectinophora gossypiella, Bupalus piniarius, Cheimatobia brumata, Lithocolletis blancardella, Hyponomeuta padella, Plutella xylostella, Malacosoma neustria, Euproctis chrysorrhoea, Lymantria spp., Bucculatrix thurberiella, Phyllocnistis citrella, Agrotis spp., Euxoa spp., Feltia spp., Earias insulana, Heliothis spp., Mamestra brassicae, Panolis flammea, Spodoptera spp., Trichoplusia ni, Carpocapsa pomonella, Pieris spp., Chilo spp., Pyrausta nubilalis, Ephestia kuehniella, Galleria mellonella, Tineola bisselliella, Tinea pellionella, Hofiannophila pseudospretella, Cacoecia podana, Capua reticulana, Choristoneura fumiferana, Clysia ambiguella, Homona magnanima, Tortrix viridana, Cnaphalocerus spp., Oulema oryzae.

From the order of the Coleoptera, for example, Anobium punctatum, Rhizopertha dominica, Bruchidius obtectus, Acanthoscelides obtectus, Hylotrupes bajulus, Agelastica alni, Leptinotarsa decemlineata, Phaedon cochleariae, Diabrotica spp., Psylliodes chrysocephala, Epilachna varivestis, Atomaria spp., Oryzaephilus surinamensis, Anthonomus spp., Sitophilus spp., Otiorrhynchus sulcatus, Cosmopolites sordidus, Ceuthorrhynchus assimilis, Hypera postica, Dermestes spp., Trogoderma spp., Anthrenus spp., Attagenus spp., Lyctus spp., Meligethes aeneus, Ptinus spp., Niptus hololeucus, Gibbium psylloides, Tribolium spp., Tenebrio molitor, Agriotes spp., Conoderus spp., Melolontha melolontha, Amphimallon solstitialis, Costelytra zealandica and Lissorhoptrus oryzophilus.

From the order of the Hymenoptera, for example, Diprion spp., Hoplocampa spp., Lasius spp., Monomorium pharaonis and Vespa spp.

From the order of the Diptera, for example, Aedes spp., Anopheles spp., Culex spp., Drosophila melanogaster, Musca spp., Fannia spp., Calliphora erythrocephala, Lucilia spp., Chrysomyia spp., Cuterebra spp., Gastrophilus spp., Hyppobosca spp., Stomoxys spp., Oestrus spp., Hypoderma spp., Tabanus spp., Tannia spp., Bibio hortulanus, Oscinella frit, Phorbia spp., Pegomyia hyoscyami, Ceratitis capitata, Dacus oleae, Tipula paludosa, Hylemyia spp. and Liriomyza spp.

From the order of the Siphonaptera, for example, Xenopsylla cheopis and Ceratophyllus spp.

From the class of the Arachnida, for example, Scorpio maurus, Latrodectus mactans, Acarus siro, Argas spp., Omithodoros spp., Dermanyssus gallinae, Eriophyes ribis, Phyllocoptruta oleivora, Boophilus spp., Rhipicephalus spp., Amblyomma spp., Hyalomma spp., Ixodes spp., Psoroptes spp., Chorioptes spp., Sarcoptes spp., Tarsonemus spp., Bryobia praetiosa, Panonychus spp., Tetranychus spp., Hemitarsonemus spp., Brevipalpus spp.

The phytoparasitic nematodes include, for example, Pratylenchus spp., Radopholus similis, Ditylenchus dipsaci, Tylenchulus semipenetrans, Heterodera spp., Globodera spp., Meloidogyne spp., Aphelenchoides spp., Longidorus spp., Xiphinema spp., Trichodorus spp., Bursaphelenchus spp.

In particular the compounds of the formula (I) according to the invention are highly active against aphids, beetles, butterfly caterpillars, spider mites and plant-damaging nematodes.

If appropriate, the compounds according to the invention can, at certain concentrations or application rates, also be used as herbicides or microbicides, for example as fungicides, antimycotics and bactericides. If appropriate, they can also be employed as intermediates or precursors for the synthesis of other active compounds.

All plants and plant parts can be treated in accordance with the invention. Plants are to be understood as meaning in the present context all plants and plant populations such as desired and undesired wild plants or crop plants (including naturally occurring crop plants). Crop plants can be plants which can be obtained by conventional plant breeding and optimization methods or by biotechnological and genetic engineering methods or by combinations of these methods, including the transgenic plants and inclusive of the plant cultivars protectable or not protectable by plant breeders' rights. Plant parts are to be understood as meaning all parts and organs of plants above and below the ground, such as shoot, leaf, flower and root, examples which may be mentioned being leaves, needles, stalks, stems, flowers, fruit bodies, fruits, seeds, roots, tubers and rhizomes. The plant parts also include harvested material, and vegetative and generative propagation material, for example cuttings, tubers, rhizomes, offsets and seeds.

Treatment according to the invention of the plants and plant parts with the active compounds is carried out directly or by allowing the compounds to act on their surroundings, environment or storage space by the customary treatment methods, for example by immersion, spraying, evaporation, fogging, scattering, painting on and, in the case of propagation material, in particular in the case of seeds, also by applying one or more coats.

The active compounds can be converted to the customary formulations, such as solutions, emulsions, wettable powders, suspensions, powders, dusts, pastes, soluble powders, granules, suspension-emulsion concentrates, natural and synthetic materials impregnated with active compound and microencapsulations in polymeric substances.

These formulations are produced in a known manner, for example by mixing the active compounds with extenders, that is, liquid solvents, and/or solid carriers, optionally with the use of surfactants, that is emulsifiers and/or dispersants, and/or foam-formers.

If the extender used is water, it is also possible to employ for example organic solvents as auxiliary solvents. Essentially, suitable liquid solvents are: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example petroleum fractions, mineral and vegetable oils, alcohols such as butanol or glycol and also their ethers and esters, ketones such as acetone, methyl ethyl ketone, methyl isobutyl ketone or cyclohexanone, strongly polar solvents such as dimethylformamide and dimethyl sulfoxide, and also water.

Suitable solid carriers are:

for example ammonium salts and ground natural minerals such as kaolins, clays, talc, chalk, quartz, attapulgite, montmorillonite or diatomaceous earth, and ground synthetic minerals, such as highly disperse silica, alumina and silicates; suitable solid carriers for granules are: for example crushed and fractionated natural rocks such as calcite, marble, pumice, sepiolite and dolomite, and also synthetic granules of inorganic and organic meals, and granules of organic material such as sawdust, coconut shells, maize cobs and tobacco stalks; suitable emulsifiers and/or foam-formers are: for example nonionic and anionic emulsifiers, such as polyoxyethylene fatty acid esters, polyoxyethylene fatty alcohol ethers, for example alkylaryl polyglycol ethers, alkylsulfonates, alkyl sulfates, arylsulfonates and also protein hydrolysates; suitable dispersants are: for example lignin-sulphite waste liquors and methylcellulose.

Tackifiers such as carboxymethylcellulose and natural and synthetic polymers in the form of powders, granules or latices, such as gum arabic, polyvinyl alcohol and polyvinyl acetate, as well as natural phospholipids such as cephalins and lecithins, and synthetic phospholipids, can be used in the formulations. Other possible additives are mineral and vegetable oils.

It is possible to use colorants such as inorganic pigments, for example iron oxide, titanium oxide and Prussian Blue, and organic dyestuffs, such as alizarin dyestuffs, azo dyestuffs and metal phthalocyanine dyestuffs, and trace nutrients such as salts of iron, manganese, boron, copper, cobalt, molybdenum and zinc.

The formulations generally comprise between 0.1 and 95% by weight of active compound, preferably between 0.5 and 90%.

The active compounds according to the invention can be employed as such or in their formulations as a mixture with known fungicides, bactericides, acaricides, nematicides or insecticides in order, for example, to increase the activity spectrum or avoid the development of resistance. In many cases synergistic effects are achieved, ie the efficacy of the mixture is greater than the efficacy of the individual components.

Suitable co-components in mixtures are, for example, the following compounds:

Fungicides:

2-phenylphenol; 8-hydroxyquinoline sulphate; acibenzolar-5-methyl; aldimorph; amidoflumet; ampropylfos; ampropylfos-potassium; andoprim; anilazine; azaconazole; azoxystrobin; benalaxyl; benodanil; benomyl; benthiavalicarbisopropyl; benzamacril; benzamacril-isobutyl; bilanafos; binapacryl; biphenyl; bitertanol; blasticidin-S; bromuconazole; bupirimate; buthiobate; butylamine; calcium polysulphide; capsimycin; captafol; captan; carbendazim; carboxin; carpropamid; carvone; chinomethionat; chlobenthiazone; chlorfenazole; chloroneb; chlorothalonil; chlozolinate; clozylacon; cyazofamid; cyflufenamid; cymoxanil; cyproconazole; cyprodinil; cyprofuram; Dagger G; debacarb; dichlofluanid; dichlone; dichlorophen; diclocymet; diclomezine; dicloran; diethofencarb; difenoconazole; diflumetorim; dimethirimol; dimethomorph; dimoxystrobin; diniconazole; diniconazole-M; dinocap; diphenylamine; dipyrithione; ditalimfos; dithianon; dodine; drazoxolon; edifenphos; epoxiconazole; ethaboxam; ethirimol; etridiazole; famoxadone; fenamidone; fenapanil; fenarimol; fenbuconazole; fenfuram; fenhexamid; fenitropan; fenoxanil; fenpiclonil; fenpropidin; fenpropimorph; ferbam; fluazinam; flubenzimine; fludioxonil; flumetover; flumorph; fluoromide; fluoxastrobin; fluquinconazole; flurprimidol; flusilazole; flusulfamide; flutolanil; flutriafol; folpet; fosetyl-Al; fosetyl-sodium; fuberidazole; furalaxyl; furametpyr; furcarbanil; furmecyclox; guazatine; hexachlorobenzene; hexaconazole; hymexazole; imazalil; imibenconazole; iminoctadine triacetate; iminoctadine tris(albesil; iodocarb; ipconazole; iprobenfos; iprodione; iprovalicarb; irumamycin; isoprothiolane; isovaledione; kasugamycin; kresoxim-methyl; mancozeb; maneb; meferimzone; mepanipyrim; mepronil; metalaxyl; metalaxyl-M; metconazole; methasulfocarb; methfuroxam; metiram; metominostrobin; metsulfovax; mildiomycin; myclobutanil; myclozolin; natamycin; nicobifen; nitrothal-isopropyl; noviflumuron; nuarimol; ofurace; orysastrobin; oxadixyl; oxolinic acid; oxpoconazole; oxycarboxin; oxyfenthiin; paclobutrazole; pefurazoate; penconazole; pencycuron; phosdiphen; phthalide; picoxystrobin; piperalin; polyoxins; polyoxorim; probenazole; prochloraz; procymidone; propamocarb; propanosine-sodium; propiconazole; propineb; proquinazid; prothioconazole; pyraclostrobin; pyrazophos; pyrifenox; pyrimethanil; pyroquilon; pyroxyfur; pyrrolenitrine; quinconazole; quinoxyfen; quintozene; simeconazole; spiroxamine; sulfur; tebuconazole; teclofialam; tecnazene; tetcyclacis; tetraconazole; thiabendazole; thicyofen; thifluzamide; thiophanate-methyl; thiram; tioxymid; tolclofos-methyl; tolylfluanid; triadimefon; triadimenol; triazbutil; triazoxide; tricyclamide; tricyclazole; tridemorph; trifloxystrobin; triflumizole; triforine; triticonazole; uniconazole; validamycin A; vinclozolin; zineb; ziram; zoxamide; (2S)-N-[2-[4-[[3-(4-chlorophenyl)-2-propynyl]oxy]-3-methoxyphenyl]ethyl]-3-methyl-2-[(methylsulfonyl)-amino]butanamide; 1-(1-naphthalenyl)-1H-pyrrole-2,5-dione; 2,3,5,6-tetrachloro-4-(methylsulfonyl)pyridine; 2-amino-4-methyl-N-phenyl-5-thiazolecarboxamide; 2-chloro-N-(2,3-dihydro-1,1,3-trimethyl-1H-inden-4-yl)-3-pyridinecarboxamide; 3,4,5-trichloro-2,6-pyridinedicarbonitrile; actinovate; cis-1-(4-chlorophenyl)-2-(1H-1,2,4-triazol-1-yl)cycloheptanol; methyl 1-(2,3-dihydro-2,2-dimethyl-1H-inden-1-yl)-1H-imidazole-5-carboxylate; monopotassium carbonate; N-(6-methoxy-3-pyridinyl)-cyclopropanecarboxamide; N-butyl-8-(1,1-dimethylethyl)-1-oxaspiro[4.5]decane-3-amine; sodium tetrathiocarbonate;

and copper salts and preparations, such as Bordeaux mixture; copper hydroxide; copper naphthenate; copper oxychloride; copper sulfate; cufraneb; copper oxide; mancopper; oxine-copper.

Bactericides:

bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulfate and other copper preparations.

Insecticides/Acaricides/Nematicides:

abamectin, ABG-9008, acephate, acequinocyl, acetamiprid, acetoprole, acrinathrin, AKD-1022, AKD-3059, AKD-3088, alanycarb, aldicarb, aldoxycarb, allethrin, allethrin 1R-isomers, alpha-cypermethrin (alphamethrin), amidoflumet, aminocarb, amitraz, avermectin, AZ-60541, azadirachtin, azamethiphos, azinphos-methyl, azinphos-ethyl, azocyclotin,

Bacillus popilliae, Bacillus sphaericus, Bacillus subtilis, Bacillus thuringiensis, Bacillus thuringiensis strain EG-2348, Bacillus thuringiensis strain GC-91, Bacillus thuringiensis strain NCTC-11821, baculoviruses, Beauveria bassiana, Beauveria tenella, bendiocarb, benfuracarb, bensultap, benzoximate, beta-cyfluthrin, beta-cypermethrin, bifenazate, bifenthrin, binapacryl, bioallethrin, bioallethrin-5-cyclopentylisomer, bioethanomethrin, biopermethrin, bioresmethrin, bistrifluron, BPMC, brofenprox, bromophos-ethyl, bromopropylate, bromfenvinfos (-methyl), BTG-504, BTG-505, bufencarb, buprofezin, butathiofos, butocarboxim, butoxycarboxim, butylpyridaben,

cadusafos, camphechlor, carbaryl, carbofuran, carbophenothion, carbosulfan, cartap, CGA-50439, chinomethionat, chlordane, chlordimeform, chloethocarb, chlorethoxyfos, chlorfenapyr, chlorfenvinphos, chlorfluazuron, chlormephos, chlorobenzilate, chloropicrin, chlorproxyfen, chlorpyrifos-methyl, chlorpyrifos (-ethyl), chlovaporthrin, chromafenozide, cis-cypermethrin, cis-resmethrin, cis-permethrin, clocythrin, cloethocarb, clofentezine, clothianidin, clothiazoben, codlemone, coumaphos, cyanofenphos, cyanophos, cycloprene, cycloprothrin, Cydia pomonella, cyfluthrin, cyhalothrin, cyhexatin, cypermethrin, cyphenothrin (1R-trans-isomer), cyromazine,

DDT, deltamethrin, demeton-5-methyl, demeton-5-methylsulfone, diafenthiuron, dialifos, diazinon, dichlofenthion, dichlorvos, dicofol, dicrotophos, dicyclanil, diflubenzuron, dimethoate, dimethylvinphos, dinobuton, dinocap, dinotefuran, diofenolan, disulfoton, docusat-sodium, dofenapyn, DOWCO-439,

eflusilanate, emamectin, emamectin-benzoate, empenthrin (1R-isomer), endosulfan, Entomopthora spp., EPN, esfenvalerate, ethiofencarb, ethiprole, ethion, ethoprophos, etofenprox, etoxazole, etrimfos,

famphur, fenamiphos, fenazaquin, fenbutatin oxide, fenfluthrin, fenitrothion, fenobucarb, fenothiocarb, fenoxacrim, fenoxycarb, fenpropathrin, fenpyrad, fenpyrithrin, fenpyroximate, fensulfothion, fenthion, fentrifanil, fenvalerate, fipronil, flonicamid, fluacrypyrim, fluazuron, flubenzimine, flubrocythrinate, flucycloxuron, flucythrinate, flufenerim, flufenoxuron, flufenprox, flumethrin, flupyrazofos, flutenzin (flufenzine), fluvalinate, fonofos, formetanate, formothion, fosmethilan, fosthiazate, fubfenprox (fluproxyfen), furathiocarb,

gamma-HCH, gossyplure, grandlure, granulosis viruses,

halfenprox, halofenozide, HCH, HCN-801, heptenophos, hexaflumuron, hexythiazox, hydramethylnone, hydroprene,

IKA-2002, imidacloprid, imiprothrin, indoxacarb, iodofenphos, iprobenfos, isazofos, isofenphos, isoprocarb, isoxathion, ivermectin,

japonilure,

kadethrin, nuclear polyhedrosis viruses, kinoprene,

lambda-cyhalothrin, lindane, lufenuron,

malathion, mecarbam, mesulfenfos, metaldehyde, metam-sodium, methacrifos, methamidophos, Metharhizium anisopliae, Metharhizium flavoviride, methidathion, methiocarb, methomyl, methoprene, methoxychlor, methoxyfenozide, metolcarb, metoxadiazone, mevinphos, milbemectin, milbemycin, MKI-245, MON-45700, monocrotophos, moxidectin, MTI-800,

naled, NC-104, NC-170, NC-184, NC-194, NC-196, niclosamide, nicotine, nitenpyram, nithiazine, NNI-0001, NNI-0101, NNI-0250, NNI-9768, novaluron, noviflumuron,

OK-5101, OK-5201, OK-9601, OK-9602, OK-9701, OK-9802, omethoate, oxamyl, oxydemeton-methyl,

Paecilomyces fumosoroseus, parathion-methyl, parathion (-ethyl), permethrin (cis-, trans-), petroleum, PH-6045, phenothrin (1R-trans isomer), phenthoate, phorate, phosalone, phosmet, phosphamidon, phosphocarb, phoxim, piperonyl butoxide, pirimicarb, pirimiphos-methyl, pirimiphos-ethyl, prallethrin, profenofos, promecarb, propaphos, propargite, propetamphos, propoxur, prothiofos, prothoate, protrifenbute, pymetrozine, pyraclofos, pyresmethrin, pyrethrum, pyridaben, pyridalyl, pyridaphenthion, pyridathion, pyrimidifen, pyriproxyfen,

quinalphos,

resmethrin, RH-5849, ribavirin, RU-12457, RU-15525,

S-421, S-1833, salithion, sebufos, SI-0009, silafluofen, spinosad, spirodiclofen, spiromesifen, sulfluramid, sulfotep, sulprofos, SZI-121,

tau-fluvalinate, tebufenozide, tebufenpyrad, tebupirimfos, teflubenzuron, tefluthrin, temephos, temivinphos, terbam, terbufos, tetrachlorvinphos, tetradifon, tetramethrin, tetramethrin (1R-isomer), tetrasul, theta-cypermethrin, thiacloprid, thiamethoxam, thiapronil, thiatriphos, thiocyclam hydrogenoxalate, thiodicarb, thiofanox, thiometon, thiosultap-sodium, thuringiensin, tolfenpyrad, tralocythrin, tralomethrin, transfluthrin, triarathene, triazamate, triazophos, triazuron, trichlophenidine, trichlorfon, triflumuron, trimethacarb, vamidothion, vaniliprole, verbutin, Verticillium lecanii,

WL-108477, WL-40027,

YI-5201, YI-5301, YI-5302,

XMC, xylylcarb,

ZA-3274, zeta-cypermethrin, zolaprofos, ZXI-8901,

the compound 3-methylphenyl propylcarbamate (Tsumacide Z),

the compound 3-(5-chloro-3-pyridinyl)-8-(2,2,2-trifluoroethyl)-8-azabicyclo[3.2.1]-octane-3-carbonitrile (CAS-Reg. No. 185982-80-3) and the corresponding 3-endo-isomer (CAS-Reg. No. 185984-60-5) (cf. WO-96/37494, WO-98/25923),

and preparations which comprise insecticidally active plant extracts, nematodes, fungi or viruses.

It is also possible to admix other known active compounds, such as herbicides, fertilizers and growth regulators.

When used as insecticides, the active compounds according to the invention can furthermore be present in their commercially available formulations and in the use forms, prepared from these formulations, as a mixture with synergistic agents. Synergistic agents are compounds which increase the action of the active compounds, without it being necessary for the synergistic agent added to be active itself.

The active compound content of the use forms prepared from the commercially available formulations can vary within wide limits. The active compound concentration of the use forms can be from 0.0000001 to 95% by weight of active compound, preferably between 0.0001 and 1% by weight.

The compounds are employed in a customary manner appropriate for the use forms.

When used against hygiene pests and pests of stored products, the active compound is distinguished by an excellent residual action on wood and clay as well as by a good stability to alkali on limed substrates.

As already mentioned above, it is possible to treat all plants and their parts according to the invention. In a preferred embodiment, wild plant species and plant cultivars, or those obtained by conventional biological breeding methods, such as crossing or protoplast fusion, and parts thereof, are treated. In a further preferred embodiment, transgenic plants and plant cultivars obtained by genetic engineering, if appropriate in combination with conventional methods (Genetic Modified Organisms), and parts thereof are treated. The term “parts” or “parts of plants” or “plant parts” has been explained above.

Particularly preferably, plants of the plant cultivars which are in each case commercially available or in use are treated according to the invention. Plant cultivars are understood as meaning plants with novel properties (“traits”) which are grown by conventional cultivation, by mutagenesis or by recombinant DNA techniques. These may be cultivars, biotypes or genotypes.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the substances and compositions to be used according to the invention, better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products are possible which exceed the effects which were actually to be expected.

The transgenic plants or plant cultivars (i.e. those obtained by genetic engineering) which are preferably to be treated according to the invention include all plants which, in the genetic modification, received genetic material which imparts particularly advantageous useful properties (“traits”) to these plants. Examples of such properties are better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products. Further and particularly emphasized examples of such properties are a better defence of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, cotton, oilseed rape and also fruit plants (with the fruits apples, pears, citrus fruits and grapes), and particular emphasis is given to maize, soya beans, potatoes, cotton and oilseed rape. Traits that are emphasized are in particular increased defence of the plants against insects by toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c Cry2Ab, Cry3Bb and CryIF and also combinations thereof) (hereinbelow referred to as “Bt plants”). Traits which are also particularly emphasized are the increased resistance of plants to fungi, bacteria and viruses by systemic acquired resistance (SAR), systemin, phytoalexins, elicitors and resistance genes and the correspondingly expressed proteins and toxins. Traits that are furthermore particularly emphasized are the increased tolerance of the plants to certain herbicidally active compounds, for example imidazolinones, sulfonylureas, glyphosate or phosphinotricin (for example the “PAT” gene). The genes which impart the desired traits in question can also be present in combination with one another in the transgenic plants. Examples of “Bt plants” which may be mentioned are maize varieties, cotton varieties, soya bean varieties and potato varieties which are sold under the trade names YIELD GARD® (for example maize, cotton, soya beans), KnockOut® (for example maize), StarLink® (for example maize), Bollgard® (cotton), Nucotn® (cotton) and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya bean), Liberty Link® (tolerance to phosphinotricin, for example oilseed rape), IMI® (tolerance to imidazolinones) and STS® (tolerance to sulfonylureas, for example maize). Herbicide-resistant plants (plants bred in a conventional manner for herbicide tolerance) which may be mentioned include the varieties sold under the name Clearfield® (for example maize). Of course, these statements also apply to plant cultivars having these genetic traits or genetic traits still to be developed, which cultivars will be developed and/or marketed in the future.

The plants listed can be treated according to the invention in a particularly advantageous manner with the compounds of the formula I or the active compound mixtures according to the invention. The preferred ranges stated above for the active compounds or mixtures also apply to the treatment of these plants. Particular emphasis is given to the treatment of plants with the compounds or mixtures specifically mentioned in the present text.

The active compounds according to the invention act not only against plant, hygiene and stored product pests, but also in the veterinary medicine sector against animal parasites (ectoparasites), such as hard ticks, soft ticks, mange mites, harvest mites, flies (biting and licking), parasitic fly larvae, lice, hair lice, feather lice and fleas. These parasites include:

From the order of the Anoplurida, for example, Haematopinus spp., Linognathus spp., Pediculus spp., Phtirus spp. and Solenopotes spp.

From the order of the Mallophagida and the suborders Amblycerina and Ischnocerina, for example, Trimenopon spp., Menopon spp., Trinoton spp., Bovicola spp., Wemeckiella spp., Lepikentron spp., Damalina spp., Trichodectes spp. and Felicola spp.

From the order of the Diptera and the suborders Nematocerina and Brachycerina, for example, Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp. and Melophagus spp.

From the order of the Siphonapterida, for example, Pulex spp., Ctenocephalides spp., Xenopsylla spp. and Ceratophyllus spp.

From the order of the Heteropterida, for example, Cimex spp., Triatoma spp., Rhodnius spp. and Panstrongylus spp.

From the order of the Blattarida, for example Blatta orientalis, Periplaneta americana, Blattela germanica and Supella spp.

From the subclass of the Acaria (acarids) and the orders of the Meta- and Mesostigmata, for example, Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp. and Varroa spp.

From the order of the Actinedida (Prostigmata) und Acaridida (Astigmata), for example, Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp. and Laminosioptes spp.

The active compounds of the formula (I) according to the invention are also suitable for controlling arthropods which infest agricultural productive livestock, such as, for example, cattle, sheep, goats, horses, pigs, donkeys, camels, buffalo, rabbits, chickens, turkeys, ducks, geese and bees, other pets, such as, for example, dogs, cats, caged birds and aquarium fish, and also so-called test animals, such as, for example, hamsters, guinea pigs, rats and mice. By controlling these arthropods, cases of death and reduction in productivity (for meat, milk, wool, hides, eggs, honey etc.) should be diminished, so that more economic and easier animal husbandry is possible by use of the active compounds according to the invention.

The active compounds according to the invention are used in the veterinary sector in a known manner by enteral administration in the form of, for example, tablets, capsules, potions, drenches, granules, pastes, boluses, the feed-through process and suppositories, by parenteral administration, such as, for example, by injection (intramuscular, subcutaneous, intravenous, intraperitoneal and the like), implants, by nasal administration, by dermal use in the form, for example, of dipping or bathing, spraying, pouring on and spotting on, washing and powdering, and also with the aid of molded articles containing the active compound, such as collars, ear marks, tail marks, limb bands, halters, marking devices and the like.

When used for cattle, poultry, pets and the like, the active compounds of the formula (I) can be used as formulations (for example powders, emulsions, free-flowing compositions), which comprise the active compounds in an amount of 1 to 80% by weight, directly or after 100 to 10 000-fold dilution, or they can be used as a chemical bath.

It has furthermore been found that the compounds according to the invention have a strong insecticidal action against insects which destroy industrial materials.

The following insects may be mentioned as examples and as preferred—but without limitation:

Beetles, such as Hylotrupes bajulus, Chlorophorus pilosis, Anobium punctatum, Xestobium rufovillosum, Ptilinus pecticornis, Dendrobium pertinex, Emobius mollis, Priobium carpini, Lyctus brunneus, Lyctus africanus, Lyctus planicollis, Lyctus linearis, Lyctus pubescens, Trogoxylon aequale, Minthes rugicollis, Xyleborus spec. Tryptodendron spec. Apate monachus, Bostrychus capucins, Heterobostrychus brunneus, Sinoxylon spec. Dinoderus minutus.

Hymenopterons, such as Sirex juvencus, Urocerus gigas, Urocerus gigas taignus, Urocerus augur.

Termites, such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis, Coptotermes formosanus.

Bristletails, such as Lepisma saccharina.

Industrial materials in the present connection are to be understood as meaning non-living materials, such as, preferably, plastics, adhesives, sizes, paper and card, leather, wood and processed wood products and coating compositions.

Wood and processed wood products are materials to be protected, especially preferably, from insect infestation.

Wood and processed wood products which can be protected by the composition according to the invention or mixtures comprising this are to be understood as meaning, for example:

building timber, wooden beams, railway sleepers, bridge components, boat jetties, wooden vehicles, boxes, pallets, containers, telegraph poles, wood panelling, wooden windows and doors, plywood, chipboard, joinery or wooden products which are used quite generally in house-building or in building joinery.

The active compounds can be used as such, in the form of concentrates or generally customary formulations, such as powders, granules, solutions, suspensions, emulsions or pastes.

The formulations mentioned can be prepared in a manner known per se, for example by mixing the active compounds with at least one solvent or diluent, emulsifier, dispersing agent and/or binder or fixing agent, a water repellent, if appropriate siccatives and UV stabilizers and if appropriate dyestuffs and pigments, and also other processing auxiliaries.

The insecticidal compositions or concentrates used for the preservation of wood and wood-derived timber products comprise the active compound according to the invention in a concentration of 0.0001 to 95% by weight, in particular 0.001 to 60% by weight.

The amount of the compositions or concentrates employed depends on the nature and occurrence of the insects and on the medium. The optimum amount employed can be determined for the use in each case by a series of tests. In general, however, it is sufficient to employ 0.0001 to 20% by weight, preferably 0.001 to 10% by weight, of the active compound, based on the material to be preserved.

Solvents and/or diluents which are used are an organic-chemical solvent or solvent mixture and/or an oily or oil-like organic-chemical solvent or solvent mixture of low volatility and/or a polar organic-chemical solvent or solvent mixture and/or water, and if appropriate an emulsifier and/or wetting agent.

Organic-chemical solvents which are preferably used are oily or oil-like solvents having an evaporation number above 35 and a flash point above 30° C., preferably above 45° C. Substances which are used as such oily or oil-like water-insoluble solvents of low volatility are appropriate mineral oils or aromatic fractions thereof, or solvent mixtures containing mineral oils, preferably white spirit, petroleum and/or alkylbenzene.

Mineral oils having a boiling range from 170 to 220° C., white spirit having a boiling range from 170 to 220° C., spindle oil having a boiling range from 250 to 350° C., petroleum and aromatics having a boiling range from 160 to 280° C., turpentine oil and the like, are advantageously employed.

In a preferred embodiment, liquid aliphatic hydrocarbons having a boiling range from 180 to 210° C. or high-boiling mixtures of aromatic and aliphatic hydrocarbons having a boiling range from 180 to 220° C. and/or spindle oil and/or monochloronaphthalene, preferably α-monochloronaphthalene, are used.

The organic oily or oil-like solvents of low volatility which have an evaporation number above 35 and a flash point above 30° C., preferably above 45° C., can be replaced in part by organic-chemical solvents of high or medium volatility, providing that the solvent mixture likewise has an evaporation number above 35 and a flash point above 30° C., preferably above 45° C., and that the insecticide/fungicide mixture is soluble or emulsifiable in this solvent mixture.

According to a preferred embodiment, some of the organic-chemical solvent or solvent mixture or an aliphatic polar organic-chemical solvent or solvent mixture is replaced. Aliphatic organic-chemical solvents containing hydroxyl and/or ester and/or ether groups, such as, for example, glycol ethers, esters or the like, are preferably used.

Organic-chemical binders which are used in the context of the present invention are the synthetic resins and/or binding drying oils which are known per se, are water-dilutable and/or are soluble or dispersible or emulsifiable in the organic-chemical solvents employed, in particular binders consisting of or comprising an acrylate resin, a vinyl resin, for example polyvinyl acetate, polyester resin, polycondensation or polyaddition resin, polyurethane resin, alkyd resin or modified alkyd resin, phenolic resin, hydrocarbon resin, such as indene-cumarone resin, silicone resin, drying vegetable oils and/or drying oils and/or physically drying binders based on a natural and/or synthetic resin.

The synthetic resin used as the binder can be employed in the form of an emulsion, dispersion or solution. Bitumen or bituminous substances can also be used as binders in an amount of up to 10% by weight. Dyestuffs, pigments, water-repelling agents, odor correctants and inhibitors or anticorrosive agents and the like which are known per se can additionally be employed.

It is preferred according to the invention for the composition or concentrate to comprise, as the organic-chemical binder, at least one alkyd resin or modified alkyd resin and/or one drying vegetable oil. Alkyd resins having an oil content of more than 45% by weight, preferably 50 to 68% by weight, are preferably used according to the invention.

All or some of the binder mentioned can be replaced by a fixing agent (mixture) or a plasticizer (mixture). These additives are intended to prevent evaporation of the active compounds and crystallization or precipitation. They preferably replace 0.01 to 30% of the binder (based on 100% of the binder employed).

The plasticizers originate from the chemical classes of phthalic acid esters, such as dibutyl, dioctyl or benzyl butyl phthalate, phosphoric acid esters, such as tributyl phosphate, adipic acid esters, such as di-(2-ethylhexyl) adipate, stearates, such as butyl stearate or amyl stearate, oleates, such as butyl oleate, glycerol ethers or higher molecular weight glycol ethers, glycerol esters and p-toluenesulfonic acid esters.

Fixing agents are based chemically on polyvinyl alkyl ethers, such as, for example, polyvinyl methyl ether or ketones, such as benzophenone or ethylenebenzophenone.

Possible solvents or diluents are, in particular, also water, if appropriate as a mixture with one or more of the abovementioned organic-chemical solvents or diluents, emulsifiers and dispersing agents.

Particularly effective preservation of wood is achieved by impregnation processes on a large industrial scale, for example vacuum, double vacuum or pressure processes.

The ready-to-use compositions can also comprise other insecticides, if appropriate, and also one or more fungicides, if appropriate.

Possible additional mixing partners are, preferably, the insecticides and fungicides mentioned in WO 94/29 268. The compounds mentioned in this document are an explicit constituent of the present application.

Especially preferred mixing partners which may be mentioned are insecticides, such as chlorpyriphos, phoxim, silafluofin, alphamethrin, cyfluthrin, cypermethrin, deltamethrin, permethrin, imidacloprid, NI-25, flufenoxuron, hexaflumuron, transfluthrin, thiacloprid, methoxyphenoxide and triflumuron,

and also fungicides, such as epoxyconazole, hexaconazole, azaconazole, propiconazole, tebuconazole, cyproconazole, metconazole, imazalil, dichlorfluanid, tolylfluanid, 3-iodo-2-propynyl butylcarbamate, N-octyl-isothiazolin-3-one and 4,5-dichloro-N-octylisothiazolin-3-one.

The compounds according to the invention can at the same time be employed for protecting objects which come into contact with saltwater or brackish water, such as hulls, screens, nets, buildings, moorings and signalling systems, against fouling.

Fouling by sessile Oligochaeta, such as Serpulidae, and by shells and species from the Ledamorpha group (goose barnacles), such as various Lepas and Scalpellum species, or by species from the Balanomorpha group (acorn barnacles), such as Balanus or Pollicipes species, increases the frictional drag of ships and, as a consequence, leads to a marked increase in operation costs owing to higher energy consumption and additionally frequent residence in the dry dock.

Apart from fouling by algae, for example Ectocarpus sp. and Ceramium sp., in particular fouling by sessile Entomostraka groups, which come under the generic term Cirripedia (cirriped crustaceans), is of particular importance.

Surprisingly, it has now been found that the compounds according to the invention, alone or in combination with other active compounds, have an outstanding antifouling action.

Using the compounds according to the invention, alone or in combination with other active compounds, allows the use of heavy metals such as, for example, in bis(trialkyltin) sulphides, tri-n-butyltin laurate, tri-n-butyltin chloride, copper(I) oxide, triethyltin chloride, tri-n-butyl(2-phenyl-4-chlorophenoxy)tin, tributyltin oxide, molybdenum disulphide, antimony oxide, polymeric butyl titanate, phenyl-(bispyridine)bismuth chloride, tri-n-butyltin fluoride, manganese ethylenebisthiocarbamate, zinc dimethyldithiocarbamate, zinc ethylenebisthiocarbamate, zinc salts and copper salts of 2-pyridinethiol 1-oxide, bisdimethyldithiocarbamoylzinc ethylenebisthiocarbamate, zinc oxide, copper(I) ethylenebisdithiocarbamate, copper thiocyanate, copper naphthenate and tributyltin halides to be dispensed with, or the concentration of these compounds to be substantially reduced.

If appropriate, the ready-to-use antifouling paints can additionally comprise other active compounds, preferably algicides, fungicides, herbicides, molluscicides, or other antifouling active compounds.

Preferably suitable components in combinations with the antifouling compositions according to the invention are:

algicides such as 2-tert-butylamino-4-cyclopropylamino-6-methylthio-1,3,5-triazine, dichlorophen, diuron, endothal, fentin acetate, isoproturon, methabenzthiazuron, oxyfluorfen, quinoclamine and terbutryn;

fungicides such as benzo[b]thiophenecarboxylic acid cyclohexylamide S,S-dioxide, dichlofluanid, fluorfolpet, 3-iodo-2-propynyl butylcarbamate, tolylfluanid and azoles such as azaconazole, cyproconazole, epoxyconazole, hexaconazole, metconazole, propiconazole and tebuconazole;

molluscicides such as fentin acetate, metaldehyde, methiocarb, niclosamid, thiodicarb and trimethacarb;

or conventional antifouling active compounds such as 4,5-dichloro-2-octyl-4-isothiazolin-3-one, diiodomethylparatryl sulfone, 2-(N,N-dimethylthiocarbamoylthio)-5-nitrothiazyl, potassium, copper, sodium and zinc salts of 2-pyridinethiol 1-oxide, pyridine-triphenylborane, tetrabutyldistannoxane, 2,3,5,6-tetrachloro-4-(methylsulfonyl)-pyridine, 2,4,5,6-tetrachloroisophthalonitrile, tetramethylthiuram disulfide and 2,4,6-trichlorophenylmaleimide.

The antifouling compositions used comprise the active compound according to the invention of the compounds according to the invention in a concentration of 0.001 to 50% by weight, in particular 0.01 to 20% by weight.

Moreover, the antifouling compositions according to the invention comprise the customary components such as, for example, those described in Ungerer, Chem. Ind. 1985, 37, 730-732 and Williams, Antifouling Marine Coatings, Noyes, Park Ridge, 1973.

Besides the algicidal, fungicidal, molluscicidal active compounds and insecticidal active compounds according to the invention, antifouling paints comprise, in particular, binders.

Examples of recognized binders are polyvinyl chloride in a solvent system, chlorinated rubber in a solvent system, acrylic resins in a solvent system, in particular in an aqueous system, vinyl chloride/vinyl acetate copolymer systems in the form of aqueous dispersions or in the form of organic solvent systems, butadiene/styrene/acrylonitrile rubbers, drying oils such as linseed oil, resin esters or modified hardened resins in combination with tar or bitumens, asphalt and epoxy compounds, small amounts of chlorine rubber, chlorinated polypropylene and vinyl resins.

If appropriate, paints also comprise inorganic pigments, organic pigments or colorants which are preferably insoluble in sea water. Paints may furthermore comprise materials such as colophonium to allow controlled release of the active compounds. Furthermore, the paints may comprise plasticizers, modifiers which affect the rheological properties and other conventional constituents. The compounds according to the invention or the abovementioned mixtures may also be incorporated into self-polishing antifouling systems.

The active compounds are also suitable for controlling animal pests, in particular insects, arachnids and mites, which are found in enclosed spaces such as, for example, dwellings, factory halls, offices, vehicle cabins and the like. They can be employed alone or in combination with other active compounds and auxiliaries in domestic insecticide products for controlling these pests. They are active against sensitive and resistant species and against all developmental stages.

These pests include:

From the order of the Scorpionidea, for example, Buthus occitanus.

From the order of the Acarina, for example, Argas persicus, Argas reflexus, Bryobia ssp., Dermanyssus gallinae, Glyciphagus domesticus, Omithodorus moubat, Rhipicephalus sanguineus, Trombicula alfreddugesi, Neutrombicula autumnalis, Dermatophagoides pteronissimus, Dermatophagoides forinae.

From the order of the Araneae, for example, Aviculariidae, Araneidae.

From the order of the Opiliones, for example, Pseudoscorpiones chelifer, Pseudoscorpiones cheiridium, Opiliones phalangium.

From the order of the Isopoda, for example, Oniscus asellus, Porcellio scaber.

From the order of the Diplopoda, for example, Blaniulus guttulatus, Polydesmus spp.

From the order of the Chilopoda, for example, Geophilus spp.

From the order of the Zygentoma, for example, Ctenolepisma spp., Lepisma saccharina, Lepismodes inquilinus.

From the order of the Blattaria, for example, Blatta orientalies, Blattella germanica, Blattella asahinai, Leucophaea maderae, Panchlora spp., Parcoblatta spp., Periplaneta australasiae, Periplaneta americana, Periplaneta brunnea, Periplaneta fuliginosa, Supella longipalpa.

From the order of the Saltatoria, for example, Acheta domesticus.

From the order of the Dermaptera, for example, Forficula auricularia.

From the order of the Isoptera, for example, Kalotermes spp., Reticulitermes spp.

From the order of the Psocoptera, for example, Lepinatus spp., Liposcelis spp.

From the order of the Coleptera, for example, Anthrenus spp., Attagenus spp., Dermestes spp., Latheticus oryzae, Necrobia spp., Ptinus spp., Rhizopertha dominica, Sitophilus granarius, Sitophilus oryzae, Sitophilus zeamais, Stegobium paniceum.

From the order of the Diptera, for example, Aedes aegypti, Aedes albopictus, Aedes taeniorhynchus, Anopheles spp., Calliphora erythrocephala, Chrysozona pluvialis, Culex quinquefasciatus, Culex pipiens, Culex tarsalis, Drosophila spp., Fannia canicularis, Musca domestica, Phlebotomus spp., Sarcophaga camaria, Simulium spp., Stomoxys calcitrans, Tipula paludosa.

From the order of the Lepidoptera, for example, Achroia grisella, Galleria mellonella, Plodia interpunctella, Tinea cloacella, Tinea pellionella, Tineola bisselliella.

From the order of the Siphonaptera, for example, Ctenocephalides canis, Ctenocephalides felis, Pulex irritans, Tunga penetrans, Xenopsylla cheopis.

From the order of the Hymenoptera, for example, Camponotus herculeanus, Lasius fuliginosus, Lasius niger, Lasius umbratus, Monomorium pharaonis, Paravespula spp., Tetramorium caespitum.

From the order of the Anoplura, for example, Pediculus humanus capitis, Pediculus humanus corporis, Phthirus pubis.

From the order of the Heteroptera, for example, Cimex hemipterus, Cimex lectularius, Rhodinus prolixus, Triatoma infestans.

In the field of household insecticides, they are used alone or in combination with other suitable active compounds, such as phosphoric acid esters, carbamates, pyrethroids, growth regulators or active compounds from other known classes of insecticides.

They are used in aerosols, pressure-free spray products, for example pump and atomizer sprays, automatic fogging systems, foggers, foams, gels, evaporator products with evaporator tablets made of cellulose or polymer, liquid evaporators, gel and membrane evaporators, propeller-driven evaporators, energy-free or passive evaporation systems, moth papers, moth bags and moth gels, as granules or dusts, in baits for spreading or in bait stations.

### PREPARATION EXAMPLES:

### Example 1

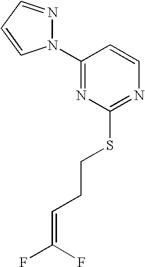 (Process (a))

At room temperature (about 20° C.), 3.0 g (12.7 mmol) of 4-chloro-2-[(4,4-difluoro-3-butenyl)-sulfanyl]-pyrimidine are, together with 9.1 g (27.9 mmol) of cesium carbonate and 0.86 g (12.7 mmol) of pyrazole, added to 60 ml of acetonitrile, and this mixture is stirred at reflux for 30 minutes. The mixture is cooled and filtered with suction, the residue is washed with acetonitrile and the solvent is distilled off. The crude product is further purified by silica gel chromatography using the mobile phase dichloromethane.

This gives 2.55 g (75% of theory) of 2-[(4,4-difluoro-3-butenyl)-sulfanyl]-4-(pyrazol-1-yl)-pyrimidine as a yellow oil.

logP (pH 2.3): 3.3

### Example 2

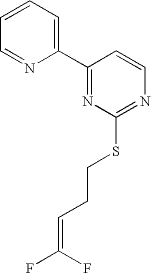 (Process (b))

5.68 g (30 mmol) of 4-(2-pyridinyl)-2-pyrimidinethiol and 16.58 g (120 mmol) of potassium carbonate are initially charged in 100 ml of acetonitrile, 5.13 g (30 mmol) of 4-bromo-1,1-difluoro-but-1-ene are added and the reaction mixture is stirred at 60° C. for 16 hours. The mixture is then concentrated under reduced pressure, the residue is distributed between water and dichloromethane, the aqueous phase is extracted two more times with dichloromethane and the combined organic phases are dried with sodium sulfate and filtered. From the filtrate, the solvent is carefully distilled off under reduced pressure.

This gives 7.6 g (88% of theory) of 2-[(4,4-difluoro-3-butenyl)-sulfanyl]-(4-(2-pyridinyl)-pyrimidine as an oil

¹H-NMR (CD₃CN): d=2.50 (m, 2H), 3.28 (t, 2H), 4.45 (m, 1H), 7.50 (m, 1H), 7.95 (m, 1H), 8.05 (m, 1H), 8.45 (m, 1H), 8.70 (m, 2H) ppm.

logP (pH 2.3)=3.33

Analogously to Examples 1 and 2, and in accordance with the general description of the preparation processes according to the invention, it is also possible to prepare, for example, the compounds of the formula (I) listed in Table 1 below.

TABLE 1
Examples of compounds of the formula (I)

| Ex. | | | | | | |
|---|---|---|---|---|---|---|
| No. | n | R | X | Y | Z | Physical data |
| 3 | 0 | H | 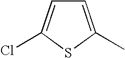 | H | H | logP = 4.73^{a)} |
| 4 | 0 | F | 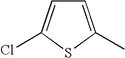 | H | H | logP = 4.70^{a)} |
| 5 | 1 | H | 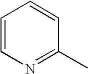 | H | H | logP = 1.47^{a)} |
| 6 | 2 | H | 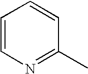 | H | H | logP = 2.08^{a)} |
| 7 | 0 | H | 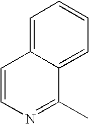 | H | H | logP = 3.67^{a)} |
| 8 | 0 | H | 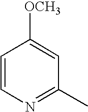 | H | H | logP = 2.41^{a)} |
| 9 | 0 | H | 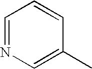 | H | H | logP = 1.96^{a)} |
| 10 | 0 | H | 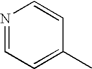 | H | H | logP = 1.72^{a)} |
| 11 | 0 | H | 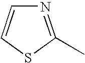 | H | H | logP = 3.33^{a)} |
| 12 | 0 | F | 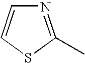 | H | H | logP = 1.56^{a)} |
| 13 | 0 | F | 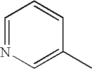 | H | H | logP = 2.05^{a)} |
| 14 | 0 | H | 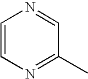 | H | H | logP = 2.91^{a)} |
| 15 | 0 | F | 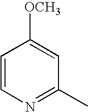 | H | H | logP = 2.54^{a)} |
| 16 | 0 | F | 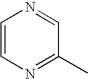 | H | H | logP = 2.92^{a)} |
| 17 | 0 | H | 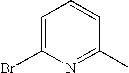 | H | H | logP = 4.51^{a)} |
| 18 | 0 | F | 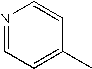 | H | H | logP = 1.77^{a)} |
| 19 | 0 | F | 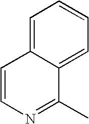 | H | H | logP = 3.69^{a)} |
| 20 | 0 | H | 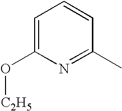 | H | H | logP = 4.91^{a)} |
| 21 | 0 | F | 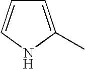 | H | H | ¹H-NMR (DMSO-D₆, δ) 11.68 ppm (NH) |
| 22 | 0 | F | 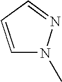 | H | H | logP = 3.27^{a)} |
| 23 | 0 | F | 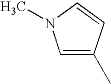 | H | H | ¹H-NMR (DMSO-D₆, δ) 3.68 ppm (N—CH₃) |
| 24 | 0 | H | 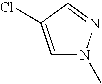 | H | H | logP = 4.13^{a)} |
| 25 | 0 | H | 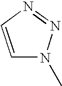 | H | H | logP = 2.62^{a)} |
| 26 | 0 | H | 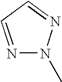 | H | H | logP = 2.70^{a)} |
| 27 | 0 | H | 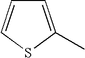 | H | H | logP = 3.83^{a)} |
| 28 | 0 | F | 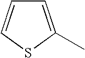 | H | H | logP = 3.84^{a)} |
| 29 | 1 | F | 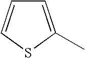 | H | H | |
| 30 | 2 | F | 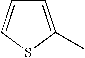 | H | H | |
| 31 | 0 | H | 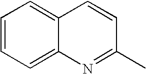 | H | H | logP = 4.81^{a)} |
| 32 | 0 | H | 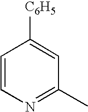 | H | H | logP = 4.77^{a)} |
| 33 | 0 | H | 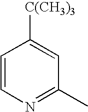 | H | H | logP = 4.80^{a)} |
| 34 | 0 | F | 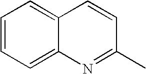 | H | H | logP = 4.81^{a)} |
| 35 | 0 | F | 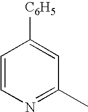 | H | H | logP = 4.76^{a)} |
| 36 | 0 | F | 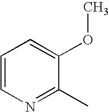 | H | H | logP = 2.25^{a)} |
| 37 | 0 | H | 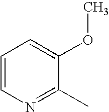 | H | H | logP = 2.14^{a)} |
| 38 | 0 | F | 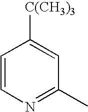 | H | H | logP = 4.81^{a)} |
| 39 | 0 | H | 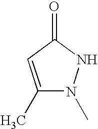 | H | H | logP = 2.55^{a)} |
| 40 | 0 | H | 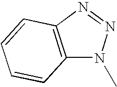 | H | H | logP = 3.97^{a)} |
| 41 | 0 | F | 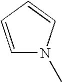 | H | H | ¹H-NMR (DMSO-D₆, δ) 2.65 ppm (m, 2H) |
| 42 | 0 | H | 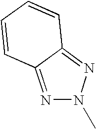 | H | H | logP = 3.98^{a)} |
| 43 | 0 | H | 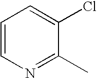 | H | H | logP = 3.32^{a)} |
| 44 | 0 | F | 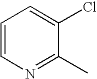 | H | H | logP = 3.35^{a)} |
| 45 | 0 | H | 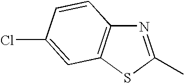 | H | H | logP = 5.57^{a)} |
| 46 | 0 | F | 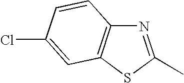 | H | H | logP = 5.49^{a)} |
| 47 | 0 | F | 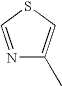 | H | H | logP = 3.18^{a)} |
| 48 | 0 | H | 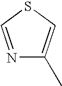 | H | H | logP = 3.14^{a)} |
| 49 | 0 | F | 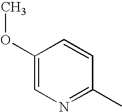 | H | H | logP = 2.17^{a)} |
| 50 | 0 | H | 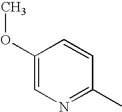 | H | H | logP = 2.12^{a)} |
| 51 | 0 | H | 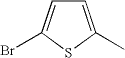 | H | H | logP = 4.77^{a)} |
| 52 | 0 | H | 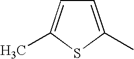 | H | H | logP = 4.30^{a)} |
| 53 | 0 | H | 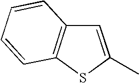 | H | H | logP = 4.87^{a)} |
| 54 | 0 | H | 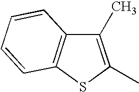 | H | H | logP = 5.25^{a)} |
| 55 | 0 | F | 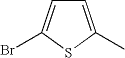 | H | H | logP = 4.73^{a)} |
| 56 | 0 | F | 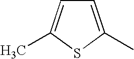 | H | H | logP = 4.31^{a)} |
| 57 | 0 | F | 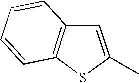 | H | H | logP = 4.85^{a)} |
| 58 | 0 | F | 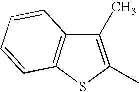 | H | H | logP = 5.20^{a)} |
| 59 | 0 | H | 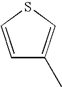 | H | H | logP = 3.80^{a)} |
| 60 | 0 | F | 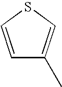 | H | H | logP = 3.82^{a)} |
| 61 | 0 | H | 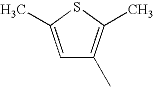 | H | H | logP = 4.79^{a)} |
| 62 | 0 | F | 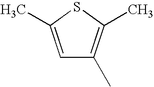 | H | H | logP = 4.79^{a)} |
| 63 | 0 | H | 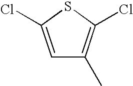 | H | H | logP = 5.65^{a)} |
| 64 | 0 | F | 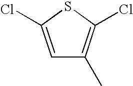 | H | H | logP = 5.55^{a)} |
| 65 | 0 | F | H | 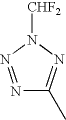 | H | |
| 66 | 0 | F | H | 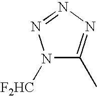 | H | |
| 67 | 0 | F | H | 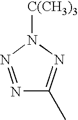 | H | logP = 4.22^{a)} |
| 68 | 2 | H | 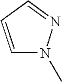 | H | H | logP = 2.01^{a)} |
| 69 | 1 | H | 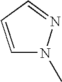 | H | H | logP = 1.55^{a)} |
| 70 | 2 | H | 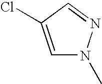 | H | H | logP = 2.60^{a)} |
| 71 | 1 | H | 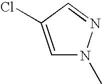 | H | H | logP = 2.09^{a)} |
| 72 | 0 | H | 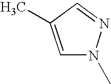 | H | H | |
| 73 | 0 | H | 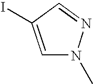 | H | H | logP = 4.34^{a)} |
| 74 | 0 | H | 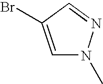 | H | H | logP = 4.24^{a)} |
| 75 | 0 | H | 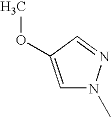 | H | H | logP = 3.47^{a)} |
| 76 | 0 | H | 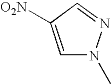 | H | H | logP = 3.48^{a)} |
| 77 | 0 | H | 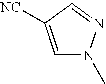 | H | H | logP = 3.22^{a)} |
| 78 | 0 | H | 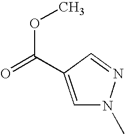 | H | H | |
| 79 | 0 | H | 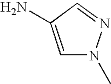 | H | H | |
| 80 | 0 | H | 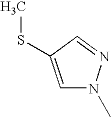 | H | H | logP = 4.00^{a)} |
| 81 | 0 | H | 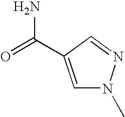 | H | H | logP = 1.98^{a)} |
| 82 | 0 | H | 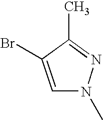 | H | H | logP '2 4.76^{a)} |
| 83 | 0 | H | 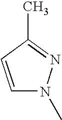 | H | H | logP = 3.64^{a)} |
| 84 | 0 | H | 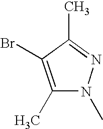 | H | H | logP = 5.25^{a)} |
| 85 | 0 | H | 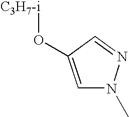 | H | H | logP = 4.26^{a)} |
| 86 | 0 | H | 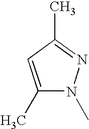 | H | H | logP = 3.95^{a)} |
| 87 | 0 | H | 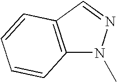 | H | H | logP = 4.26^{a)} |
| 88 | 0 | H | 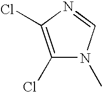 | H | H | logP = 3.57^{a)} |
| 89 | 0 | H | 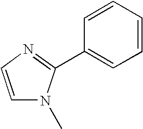 | H | H | logP = 1.99^{a)} |
| 90 | 0 | H | 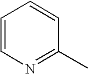 | H | H | logP = 3.14^{a)} |
| 91 | 0 | H | 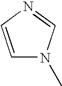 | H | H | logP = 1.46^{a)} |
| 92 | 0 | H | 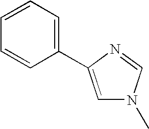 | H | H | logP = 3.45^{a)} |
| 93 | 0 | H | 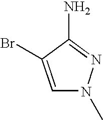 | H | H | logP = 3.15^{a)} |
| 94 | 0 | H | 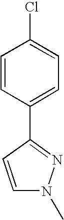 | H | H | logP = 5.69^{a)} |
| 95 | 0 | H | 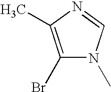 | H | H | logP = 3.21^{a)} |
| 96 | 0 | H | 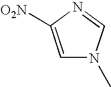 | H | H | logP = 2.63^{a)} |
| 97 | 0 | H | 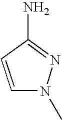 | H | H | logP = 2.31^{a)} |
| 98 | 0 | H | 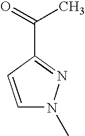 | H | H | logP = 3.47^{a)} |
| 99 | 0 | H | 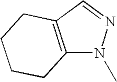 | H | H | logP = 4.64^{a)} |
| 100 | 0 | H | 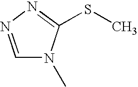 | H | H | logP = 3.39^{a)} |
| 101 | 0 | H | 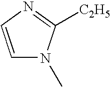 | H | H | logP = 1.45^{a)} |
| 102 | 0 | H | 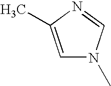 | H | H | logP = 1.48^{a)} |
| 103 | 0 | H | 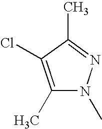 | H | H | logP = 5.13^{a)} |
| 104 | 0 | H | 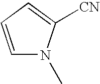 | H | H | logP = 3.23^{a)} |
| 105 | 0 | H | 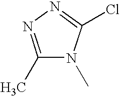 | H | H | logP = 3.49^{a)} |
| 106 | 0 | H | 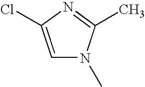 | H | H | logP = 2.99^{a)} |
| 107 | 0 | H | 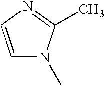 | H | H | logP = 1.30^{a)} |
| 108 | 0 | H | 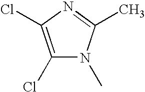 | H | H | logP '2 3.59^{a)} |
| 109 | 0 | H | 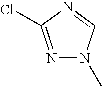 | H | H | logP = 3.31^{a)} |
| 110 | 0 | H | 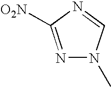 | H | H | logP = 2.95^{a)} |
| 111 | 0 | H | 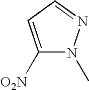 | H | H | logP = 3.09^{a)} |
| 112 | 0 | H | 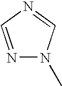 | H | H | logP = 2.44^{a)} |
| 113 | 0 | H | 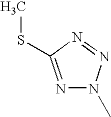 | H | H | logP = 3.34^{a)} |
| 114 | 0 | H | 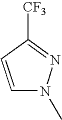 | H | H | logP = 4.41^{a)} |
| 115 | 0 | H | 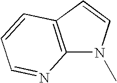 | H | H | logP = 4.60^{a)} |
| 116 | 0 | H | 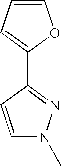 | H | H | logP = 4.22^{a)} |
| 117 | 0 | H | 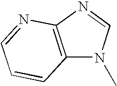 | H | H | logP '2 2.22^{a)} |
| 118 | 0 | H | 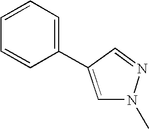 | H | H | logP = 4.71^{a)} |
| 119 | 0 | H | 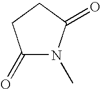 | H | H | ¹H-NMR (DMSO-D₆, δ: 8.80-8.81 ppm |
| 120 | 0 | H | 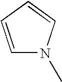 | H | H | ¹H-NMR (DMSO-D₆, δ): 3.21 ppm |
| 121 | 0 | H | 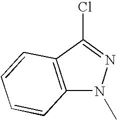 | H | H | ¹H-NMR (DMSO-D₆, δ): 3.33 ppm |
| 122 | 0 | H | 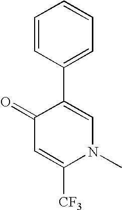 | H | H | logP = 4.49^{a)} |
| 123 | 0 | H | 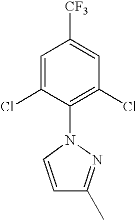 | H | H | logP = 5.21^{a)} |
| 124 | 0 | F | 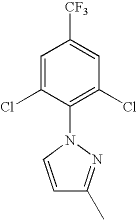 | H | H | ¹H-NMR (DMSO-D₆, δ): 8.65-8.76 ppm |
| 125 | 0 | H | 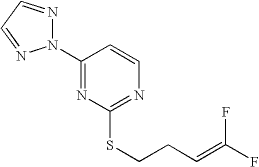 | H | H | ¹H-NMR (DMSO-D₆, δ): 4.58-4.67 ppm |
| 126 | 0 | H | 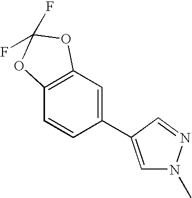 | H | H | logP = 5.20^{a)} |

The logP values given in the table were determined in accordance with EEC Directive 79/831 Annex V.A8 by HPLC (High Performance Liquid Chromatography) using a reversed-phase column (C 18). Temperature: 43° C.

(a) Mobile phases for the determination of the acidic range: 0.1% aqueous phosphoric acid, acetonitrile; linear gradient from 10% acetonitrile to 90% acteonitrile—the corresponding measurement results in Table 1 are marked^{a}).

(b) Mobile phases for the determination of the neutral range: 0.01 molar aqueous phosphate buffer solution, acetonitrile; linear gradient from 10% acetonitrile to 90% acetonitrile—the corresponding measurement results in Table 1 are marked^{b}).

Calibration was carried out using unbranched alkan-2-ones (having 3 to 16 carbon atoms) with known logP values (determination of the logP values by the retention times using linear interpolation between two successive alkanones).

The lambda max values were determined in the maxima of the chromatographic signals using the UV spectra from 200 nm to 400 nm.

Starting Materials of the Formula (II):

### Example (II-1)

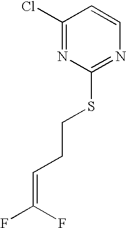 Step 1

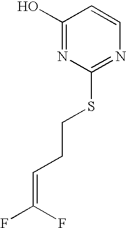

22.5 g (175 mmol) of 4-hydroxy-2-mercapto-pyrimidine are suspended in 150 ml of ethanol and 150 ml of water, and a solution of 7.0 g of sodium hydroxide in 20 ml of ethanol and 20 ml of water is added at room temperature (about 20° C.). 33.0 g (193 mmol) of 4-bromo-1,1-difluoro-1-butene are then added dropwise, and the mixture is stirred at 60° C. for 30 minutes. After cooling, the mixture is stirred at 0° C. for a further 3 hours, and the resulting precipitate is filtered off with suction. The aqueous phase is extracted with t-butyl methyl ether and the organic phase is dried with sodium sulfate and filtered. From the filtrate, the solvent is carefully distilled off under reduced pressure. This gives a yellow solid (12.2 g=32% of theory as first fraction of the desired product). The aqueous mother liquor is saturated with sodium chloride and extracted four times with in each case 100 ml of ethyl acetate. The combined organic phases are dried with sodium sulfate and filtered. From the filtrate, the solvent is carefully distilled off under reduced pressure. This gives a yellow solid as second fraction of the desired product. The combined fractions are recrystallized from t-butyl methyl ether.

Total yield: 21.1 g (51% of theory) of 4-hydroxy-2-[(4,4-difluoro-3-butenyl)-sulfanyl]-pyrimidine (melting point: 78° C.).
Step 2

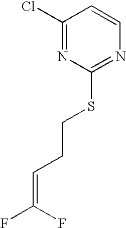

At room temperature (about 20° C.), 5.5 g (25.2 mmol) of 4-hydroxy-2-[(4,4-difluoro-3-butenyl)-sulfanyl]-pyrimidine (=2-[(4,4-difluoro-3-butenyl)-sulfanyl]-4-pyrimidinol) are added a little at a time to 38.6 g (23.5 ml, 252 mmol) of phosphorus oxychloride, and the mixture is, under reflux, heated at the boil for 10 minutes. The mixture is then concentrated to 3/4 using a rotary evaporator, the oily residue is added dropwise to 50 ml of ice-water, with cooling, the pH is adjusted to 5-6 using 2N aqueous sodium hydroxide solution and the mixture is extracted repeatedly with dichloromethane. The organic solvent is distilled off under reduced pressure, giving the product in the form of a yellow oil.

Yield: 5.5 g (88% of theory)

logP (pH 2.3): 3.3

Use Examples:

### Example A

Aphis gossypii Test

Solvent: 7 parts by weight of dimethylformamide
Emulsifier: 2 parts by weight of alkylaryl polyglycol ether

To produce a suitable preparation of active compound, 1 part by weight of active compound is mixed with the stated amounts of solvent and emulsifier, and the concentrate is diluted with emulsifier-containing water to the desired concentration.

Cotton leaves (Gossypium hirsutum) which are heavily infested by the cotton aphid (Aphis gossypii) are treated by being dipped into the preparation of active compound of the desired concentration.

After the desired period of time, the kill in % is determined. 100% means all aphids have been killed; 0% means that none of the aphids have been killed.

Active compounds, active compound concentrations and test results are shown in Table A.

TABLE A
Plant-damaging insects
Aphis gossipii test

| | Concentration of active | Kill rate in % |
|---|---|---|
| Active compounds | compound in ppm | after 6^{d} |
| 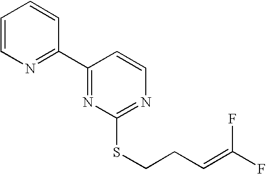 | | |
| (2) | 500 | 99 |
| 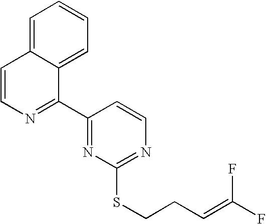 | | |
| (7) | 500 | 98 |
| 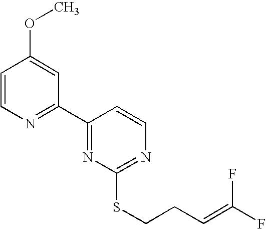 | | |
| (8) | 500 | 99 |
| 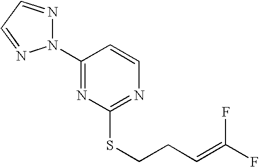 | | |
| (26) | 500 | 95 |

### Example B

Heliothis armigera Test

Solvent: 7 parts by weight of dimethylformamide
Emulsifier: 2 parts by weight of alkylaryl polyglycol ether

To produce a suitable preparation of active compound, 1 part by weight of active compound is mixed with the stated amounts of solvent and emulsifier, and the concentrate is diluted with emulsifier-containing water to the desired concentration.

Soya bean shoots (Glycine max) are treated by being dipped into the preparation of active compound of the desired concentration and are populated with Heliothis annigera caterpillars while the leaves are still moist.

After the desired period of time, the kill in % is determined. 100% means that all caterpillars have been killed; 0% means that none of the caterpillars have been killed.

Active compounds, active compound concentrations and test results are shown in Table B.

TABLE B
Plant-damaging insects
Heliothis armigera test

| | Concentration of active | Kill rate in % |
|---|---|---|
| Active compounds | compound in ppm | after 7^{d} |
| 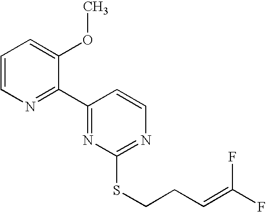 | | |
| (37) | 500 | 100 |
| 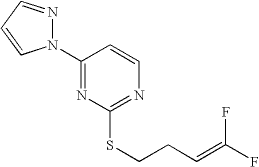 | | |
| (1) | 500 | 100 |
| 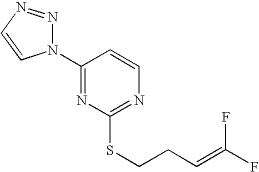 | | |
| (25) | 500 | 100 |

### Example C

Meloidogyne Test

Solvent: 7 parts by weight of dimethylformamide
Emulsifier: 2 parts by weight of alkylaryl polyglycol ether

To produce a suitable preparation of active compound, 1 part by weight of active compound is mixed with the stated amounts of solvent and emulsifier, and the concentrate is diluted with water to the desired concentration.

Vessels are filled with sand, solution of active compound, Meloidogyne incognita egg/larvae suspension and lettuce seeds. The lettuce seeds germinate and the plants develop. On the roots, galls are formed.

After the desired period of time, the nematicidal action is determined in % using the formation of galls. 100% means that no galls were found; 0% means that the number of galls on the treated plants corresponds to that of the untreated control.

Active compounds, active compound concentrations and test results are shown inter alia in Table C. The compounds with Example numbers 82, 85, 86, 87, 88, 89, 90, 91, 92, 93, 94, 95, 96, 97, 98, 99, 100, 101, 102, 103, 104, 106, 107, 108, 109, 110, 111, 112, 114, 115, 116, 117, 118, 119, 120, 121, 122, 123, 124 and 125 also achieve a kill rate of 100% in the test described above at a concentration of 20 ppm.

TABLE C
Plant-damaging nematodes
Meloidogyne test

| | Concentration of active | Kill rate in % |
|---|---|---|
| Active compounds | compound in ppm | after 14^{d} |
| 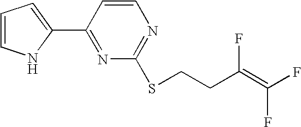 | | |
| (21) | 20 | 100 |
| 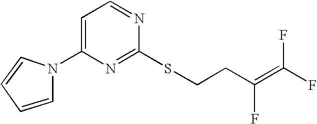 | | |
| (41) | 20 | 100 |
| 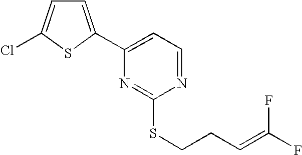 | | |
| (3) | 20 | 100 |
| 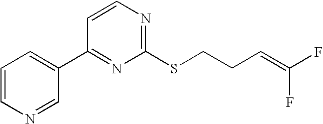 | | |
| (9) | 20 | 100 |
| 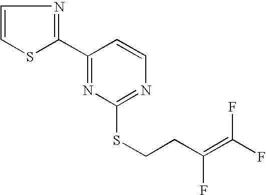 | | |
| (12) | 20 | 100 |
| 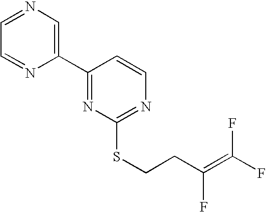 | | |
| (16) | 20 | 100 |
| 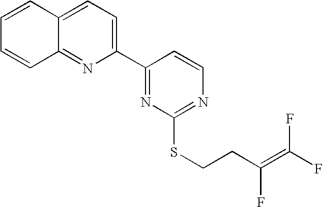 | | |
| (34) | 20 | 100 |
| 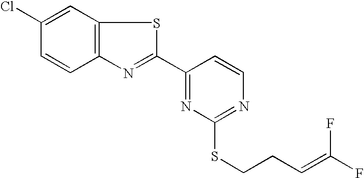 | | |
| (45) | 20 | 100 |
| 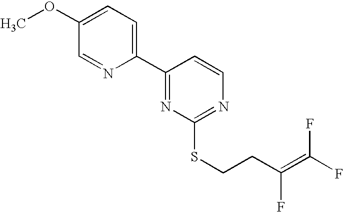 | | |
| (49) | 20 | 100 |
| 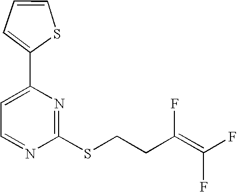 | | |
| (28) | 20 | 100 |
| 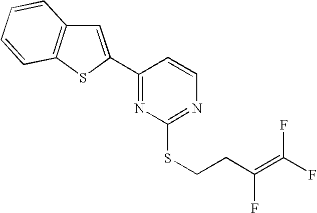 | | |
| (57) | 20 | 100 |
| 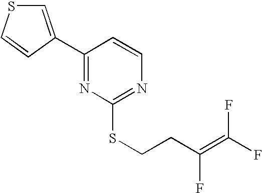 | | |
| (60) | 20 | 100 |
| 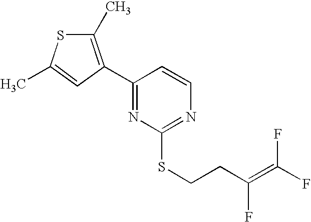 | | |
| (62) | 20 | 100 |
| 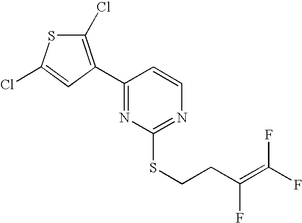 | | |
| (64) | 20 | 100 |
| 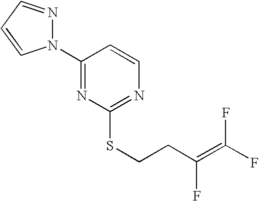 | | |
| (22) | 20 | 100 |
| 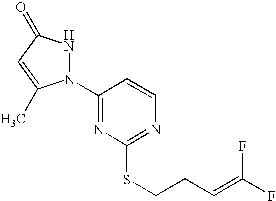 | | |
| (39) | 20 | 100 |
| 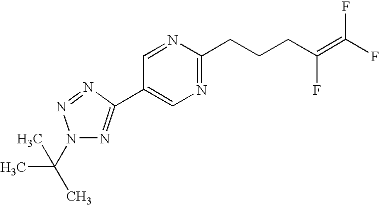 | | |
| (67) | 20 | 100 |

### Example D

Myzus Test

Solvent: 7 parts by weight of dimethylformamide
Emulsifier: 2 parts by weight of alkylaryl polyglycol ether

To produce a suitable preparation of active compound, 1 part by weight of active compound is mixed with the stated amounts of solvent and emulsifier, and the concentrate is diluted with emulsifier-containing water to the desired concentration.

Cabbage leaves (Brassica oleracea) which are heavily infested by the peach aphid (Myzus persicae) are treated by being dipped into the preparation of active compound of the desired concentration.

After the desired period of time, the kill in % is determined. 100% means that all aphids have been killed; 0% means that none of the aphids have been killed.

Active compounds, active compound concentrations and test results are shown in Table D.

TABLE D
Plant-damaging insects
Myzus test

| | Concentration of active | Kill rate in % |
|---|---|---|
| Active compounds | compound in ppm | after 6^{d} |
| 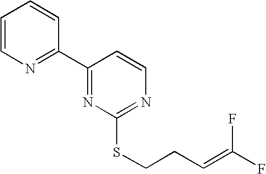 | | |
| (2) | 500 | 100 |
| 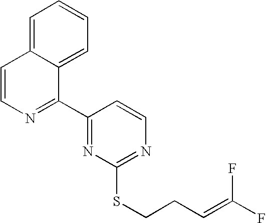 | | |
| (7) | 500 | 100 |
| 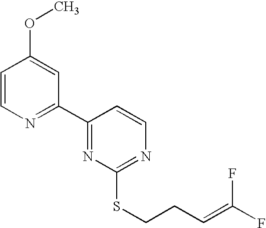 | | |
| (8) | 500 | 95 |
| 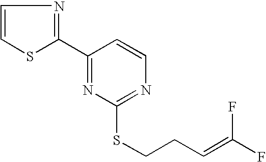 | | |
| (11) | 500 | 95 |
| 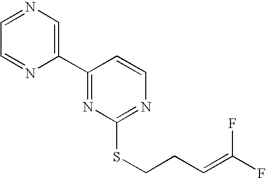 | | |
| (14) | 500 | 95 |
| 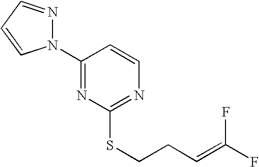 | | |
| (1) | 500 | 99 |
| 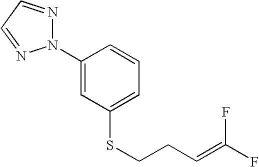 | | |
| (26) | 500 | 99 |

### Example E

Phaedon larvae Test

Solvent: 7 parts by weight of dimethylformamide
Emulsifier: 2 parts by weight of alkylaryl polyglycol ether

To produce a suitable preparation of active compound, 1 part by weight of active compound is mixed with the stated amounts of solvent and emulsifier, and the concentrate is diluted with emulsifier-containing water to the desired concentration.

Cabbage leaves (Brassica oleracea) are treated by being dipped into the preparation of active compound of the desired concentration and are populated with larvae of the mustard beetle (Phaedon cochleariae) while the leaves are still moist.

After the desired period of time, the kill in % is determined. 100% means that all beetle larvae have been killed; 0% means that none of the beetle larvae have been killed.

Active compounds, active compound concentrations and test results are shown in Table E.

TABLE E
Plant-damaging insects
Phaedon larvae test

| | Concentration of active | Kill rate in % |
|---|---|---|
| Active compounds | compound in ppm | after 7^{d} |
| 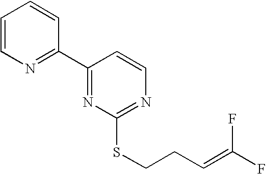 | | |
| (2) | 500 | 100 |
| 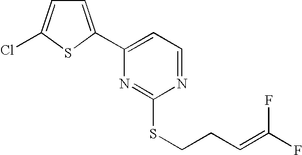 | | |
| (3) | 500 | 100 |
| 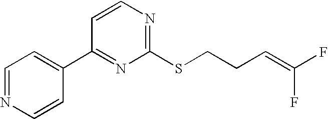 | | |
| (10) | 500 | 100 |
| 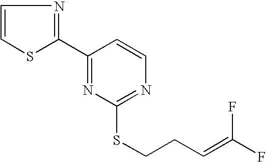 | | |
| (11) | 500 | 95 |
| 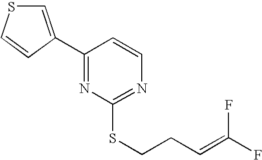 | | |
| (59) | 500 | 100 |
| 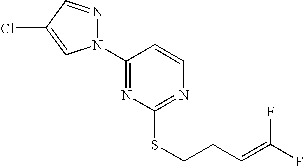 | | |
| (24) | 500 | 100 |
| 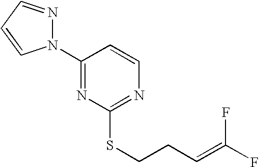 | | |
| (1) | 500 | 100 |
| 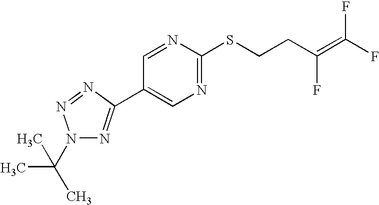 | | |
| (67) | 500 | 100 |

### Example F

Plutella Test

Solvent: 7 parts by weight of dimethylformamide
Emulsifier: 2 parts by weight of alkylaryl polyglycol ether

To produce a suitable preparation of active compound, 1 part by weight of active compound is mixed with the stated amounts of solvent and emulsifier, and the concentrate is diluted with emulsifier-containing water to the desired concentration.

Cabbage leaves (Brassica oleracea) are treated by being dipped into the preparation of active compound of the desired concentration and are populated with caterpillars of the diamondback moth (Plutella xylostella) while the leaves are still moist.

After the desired period of time, the kill in % is determined. 100% means that all caterpillars have been killed; 0% means that none of the caterpillars have been killed.

Active compounds, active compound concentrations and test results are shown in Table F.

TABLE F
Plant-damaging insects
Plutella test

| | | |
|---|---|---|
| | Concentration of active | Kill rate in % |
| Active compounds | compound in ppm | after 7^{d} |

| | | |
|---|---|---|
| 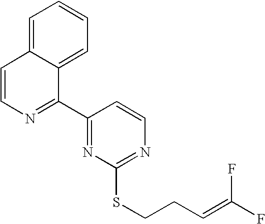 | | |
| (7) | 500 | 100 |
| 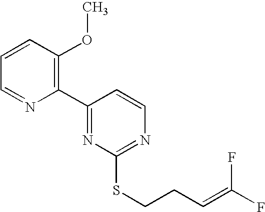 | | |
| (37) | 500 | 100 |
| 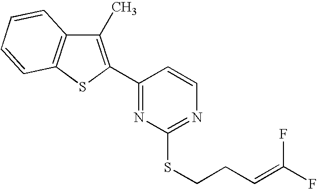 | | |
| (54) | 500 | 100 |
| 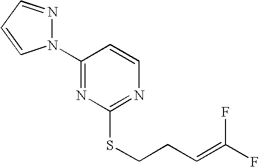 | | |
| (1) | 500 | 100 |
| 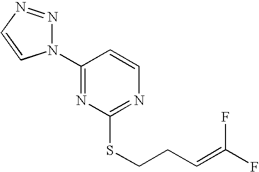 | | |
| (25) | 500 | 100 |

### Example G

Spodoptera frugiperda Test

Solvent: 7 parts by weight of dimethylformamide
Emulsifier: 2 parts by weight of alkylaryl polyglycol ether

To produce a suitable preparation of active compound, 1 part by weight of active compound is mixed with the stated amounts of solvent and emulsifier, and the concentrate is diluted with emulsifier-containing water to the desired concentration.

Cabbage leaves (Brassica oleracea) are treated by being dipped into the preparation of active compound of the desired concentration and are populated with caterpillars of the armyworm (Spodoptera frugiperda) while the leaves are still moist.

After the desired period of time, the kill in % is determined. 100% means that all caterpillars have been killed; 0% means that none of the caterpillars have been killed.

Active compounds, active compound concentrations and test results are shown in Table G.

TABLE G
Plant-damaging insects
Spodoptera frugiperda test

| | Concentration of active | Kill rate in % |
|---|---|---|
| Active compounds | compound in ppm | after 7^{d} |
| 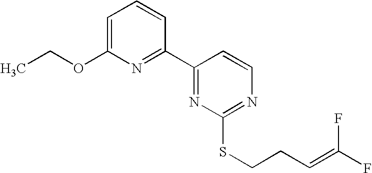 | | |
| (20) | 500 | 100 |
| 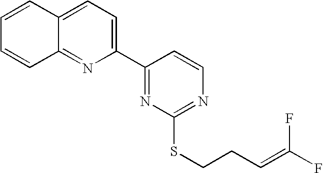 | | |
| (31) | 500 | 100 |
| 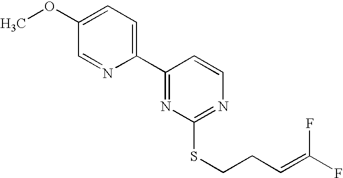 | | |
| (50) | 500 | 100 |
| 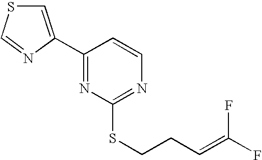 | | |
| (48) | 500 | 100 |
| 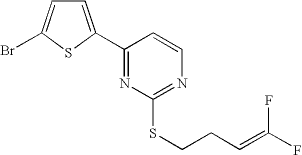 | | |
| (51) | 500 | 100 |
| 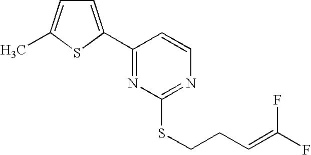 | | |
| (52) | 500 | 100 |
| 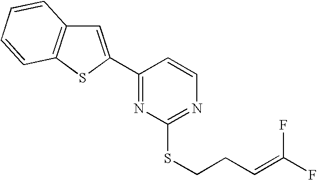 | | |
| (53) | 500 | 100 |
| 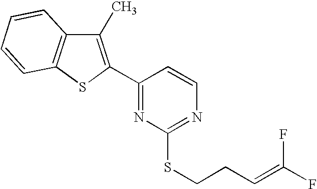 | | |
| (54) | 500 | 100 |
| 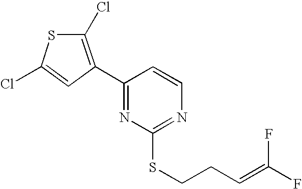 | | |
| (63) | 500 | 100 |
| 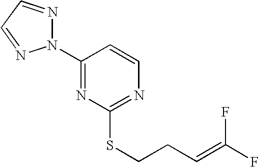 | | |
| (26) | 500 | 100 |
| 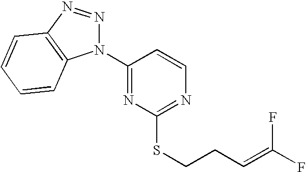 | | |
| (40) | 500 | 100 |

### Example H

Tetranychus Test (OP-Resistant/Dip Treatment)

Solvent: 7 parts by weight of dimethylformamide
Emulsifier: 2 parts by weight of alkylaryl polyglycol ether

To produce a suitable preparation of active compound, 1 part by weight of active compound is mixed with the stated amounts of solvent and emulsifier, and the concentrate is diluted with emulsifier-containing water to the desired concentration.

Bean plants (Phaseolus vulgaris) which are heavily infested by all stages of the greenhouse red spider mite (Tetranychus urticae) are dipped into a preparation of active compound of the desired concentration.

After the desired period of time, the effect in % is determined. 100% means that all spider mites have been killed; 0% means that none of the spider mites have been killed.

Active compounds, active compound concentrations and test results are shown in Table H.

TABLE H
Plant-damaging mites
Tetranychus test (OP-resistant/dip treatment)

| | Concentration of active | Kill rate in % |
|---|---|---|
| Active compounds | compound in ppm | after 7^{d} |
| 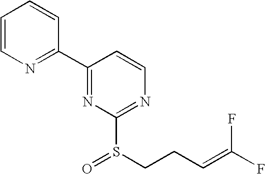 | | |
| (5) | 100 | 100 |
| 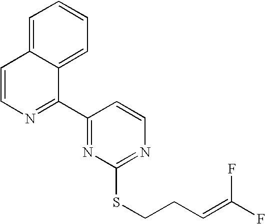 | | |
| (7) | 100 | 100 |
| 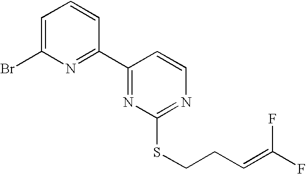 | | |
| (17) | 100 | 100 |
| 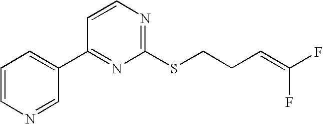 | | |
| (9) | 100 | 100 |
| 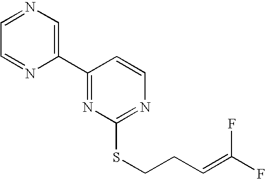 | | |
| (14) | 100 | 100 |
| 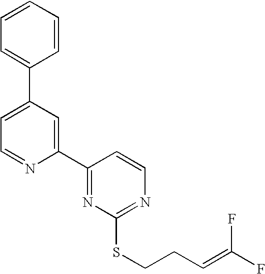 | | |
| (32) | 100 | 100 |
| 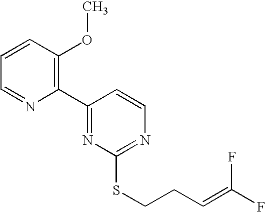 | | |
| (37) | 100 | 100 |
| 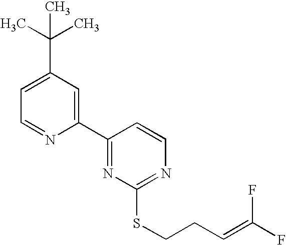 | | |
| (33) | 100 | 100 |
| 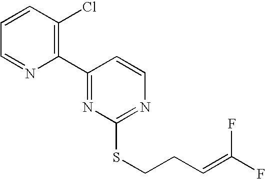 | | |
| (43) | 100 | 99 |
| 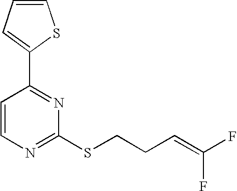 | | |
| (27) | 100 | 100 |
| 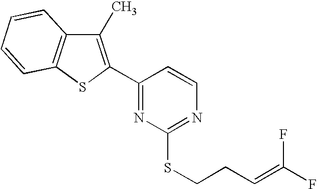 | | |
| (54) | 100 | 100 |
| 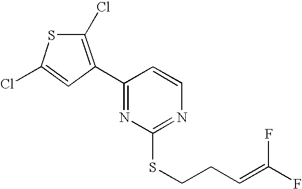 | | |
| (63) | 100 | 100 |
| 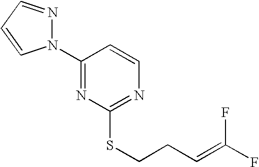 | | |
| (1) | 100 | 100 |
| 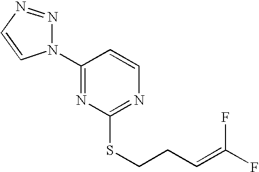 | | |
| (25) | 100 | 100 |
| 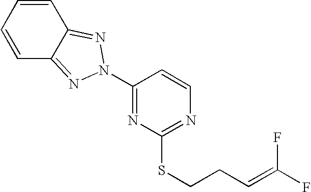 | | |
| (42) | 100 | 99 |

1-10. (canceled)

11. A compound of formula (I) 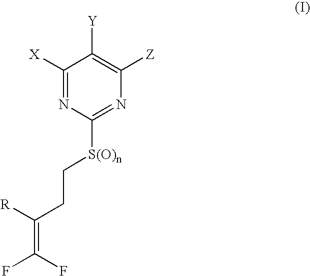
in which
n represents the numbers 0, 1, or 2,
R represents hydrogen or fluorine,
X, Y, and Z independently of one another represent hydrogen, nitro, cyano, carboxyl, carbamoyl, thiocarbamoyl, hydroxyl, cyanato, thiocyanato, or halogen; represent optionally cyano-, halogen- or C₁-C₆-alkoxy-substituted alkyl, alkylcarbonyl, alkoxy, alkoxycarbonyl, alkylthio, alkylsulfinyl, alkylsulfonyl, alkylsulfonyloxy, alkylamino, alkylaminocarbonyl, alkoxycarbonylamino, alkylsulfonylamino, dialkylamino, dialkylaminocarbonyl, or dialkylaminosulfonyl having in each case 1 to 6 carbon atoms in the alkyl groups; represent optionally halogen-substituted alkenyl, alkenyloxy, alkenyloxycarbonyl, alkynyl, alkynyloxy, or alkynyloxycarbonyl having in each case 2 to 6 carbon atoms in the alkenyl or alkynyl groups; represent cycloalkyl, cycloalkenyl, or cycloalkylalkyl having in each case 3 to 6 carbon atoms in the cycloalkyl or cycloalkenyl group and, if appropriate, 1 to 4 carbon atoms in the alkyl moiety; or represent phenyl, phenoxy, phenylthio, pyridyl, furyl, or thienyl, each of which is optionally substituted by nitro, cyano, carboxyl, carbamoyl, thiocarbamoyl, halogen, C₁-C₄-alkyl, C₁-C₄-haloalkyl, C₁-C₄-alkoxy, C₁-C₄-haloalkoxy, C₁-C₄-alkylthio, C₁-C₄-haloalkylthio, or C₁-C₄-alkoxycarbonyl; or
X, Y, and Z together represent a benzo grouping or represent alkylene or alkenylene having in each case up to 4 carbon atoms, where the carbon chain may be interrupted by 1 to 3 nitrogen atoms or 1 to 2 not directly adjacent oxygen atoms, wherein the ring thus formed is optionally substituted by halogen or alkyl having 1 to 4 carbon atoms, and at least one of the radicals X, Y, or Z represents a saturated or unsaturated monocyclic or bicyclic heterocyclyl grouping having up to 10 carbon atoms and up to 5 nitrogen atoms and/or one oxygen or sulfur atom and optionally additionally containing one or two oxo groupings (C═O), a thioxo grouping (C═S), a —SO grouping, or a —SO₂ grouping and that optionally contains up to 4 substituents selected from the group consisting of nitro; amino; cyano; carboxyl; carbamoyl; thiocarbamoyl; hydroxyl; halogen; optionally cyano-, halogen-, or C₁-C₆-alkoxy-substituted alkyl, alkylcarbonyl, alkoxy, alkoxycarbonyl, alkylthio, alkylsulfinyl, alkylsulfonyl, alkylsulfonyloxy, alkylamino, alkylaminocarbonyl, alkoxycarbonylamino, alkylsulfonylamino, dialkylamino, dialkylaminocarbonyl, or dialkylaminosulfonyl having in each case 1 to 6 carbon atoms in the alkyl groups; optionally halogen-substituted alkenyl, alkenyloxy, alkenyloxycarbonyl, alkynyl, alkynyloxy, or alkynyloxycarbonyl having in each case 2 to 6 carbon atoms in the alkenyl or alkynyl groups; cycloalkyl, cycloalkenyl, or cycloalkylalkyl having in each case 3 to 6 carbon atoms in the cycloalkyl or cycloalkenyl group and, if appropriate, 1 to 4 carbon atoms in the alkyl moiety; and phenyl, benzyl, phenoxy, phenylthio, pyridyl, furyl, or thienyl, each of which is optionally substituted by nitro, cyano, carboxyl, carbamoyl, thiocarbamoyl, halogen, C₁-C₄-alkyl, C₁-C₄-haloalkyl, C₁-C₄-alkoxy, C₁-C₄-haloalkoxy, C₁-C₄-alkylthio, C₁-C₄-haloalkylthio, or C₁-C₄-alkoxycarbonyl.

12. A process for preparing compounds of formula (I) as claimed in claim 11 comprising
(a) reacting a compound of formula (II) 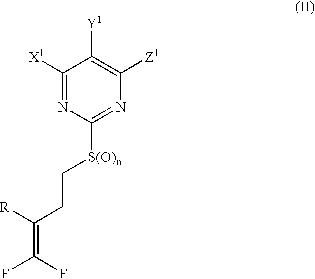
in which
n and R have the meanings given for formula (I) in claim 11, and
X¹, Y¹, and Z¹ each have the meanings given for formula (I) in claim 11 for the corresponding radicals X, Y, and Z, with the provisos that X¹, Y¹, and Z¹ do not represent a heterocyclyl grouping and at least one of the radicals X¹, Y¹, or Z¹ represents halogen,
with a heterocycle of formula (IIIa), (IIIb), or (IIIc)
H—X² (IIIa),
H—Y² (IIIb),
or
H-Z² (IIIc)
in which
X², Y² and Z² represent a saturated or unsaturated, monocyclic or bicyclic heterocyclyl grouping having up to 10 carbon atoms and up to 5 nitrogen atoms and/or one oxygen or sulfur atom and optionally additionally containing an oxo grouping (C═O), a thioxo grouping (C═S), a —SO grouping, or a —SO₂ grouping and optionally containing up to 4 substituents selected from the group consisting of nitro; amino; cyano; carboxyl; carbamoyl; thiocarbamoyl; hydroxyl; halogen; optionally cyano-, halogen-, or C₁-C₆-alkoxy-substituted alkyl, alkylcarbonyl, alkoxy, alkoxycarbonyl, alkylthio, alkylsulfinyl, alkylsulfonyl, alkylsulfonyloxy, alkylamino, alkylaminocarbonyl, alkoxycarbonylamino, alkylsulfonylamino, dialkylamino, dialkylaminocarbonyl, or dialkylaminosulfonyl having in each case 1 to 6 carbon atoms in the alkyl groups; optionally halogen-substituted alkenyl, alkenyloxy, alkenyloxycarbonyl, alkynyl, alkynyloxy, or alkynyloxycarbonyl having in each case 2 to 6 carbon atoms in the alkenyl or alkynyl groups; cycloalkyl, cycloalkenyl, or cycloalkylalkyl having in each case 3 to 6 carbon atoms in the cycloalkyl or cycloalkenyl group and, if appropriate, 1 to 4 carbon atoms in the alkyl moiety; and phenyl, benzyl, phenoxy, phenylthio, pyridyl, furyl, or thienyl, each of which is optionally substituted by nitro, cyano, carboxyl, carbamoyl, thiocarbamoyl, halogen, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkoxy-carbonyl, C₁-C₂-alkylenedioxy, or C₁-C₂-haloalkylenedioxy,
optionally in the presence of one or more reaction auxiliaries and optionally in the presence of one or more diluents, or
(b) reacting a compound of formula (IV), 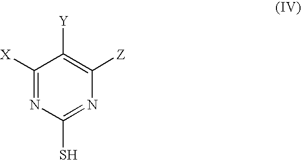
in which X, Y, and Z have the meanings given for formula (I) in claim 11,
with a compound of formula (V), 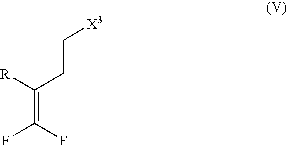
in which
R has the meanings given for formula (I) in claim 11, and
X³ represents halogen,
optionally in the presence of one or more reaction auxiliaries and optionally in the presence of one or more diluents.

13. A process according to claim 12 additionally comprising converting a compound of formula (I) obtained according to process (a) or (b) are in a into one or more other compound of formula (I).

14. A compound of formula (I) according to claim 11 in which
n represents the numbers 0 or 2,
R represents hydrogen or fluorine, and
X, Y, and Z independently of one another represent hydrogen, nitro, cyano, carboxyl, carbamoyl, thiocarbamoyl, hydroxyl, cyanato, thiocyanato, or halogen; represent optionally cyano-, fluorine-, chlorine-, bromine-, or C₁-C₄-alkoxy-substituted alkyl, alkylcarbonyl, alkoxy, alkoxycarbonyl, alkylthio, alkylsulfinyl, alkylsulfonyl, alkylsulfonyloxy, alkylamino, alkylaminocarbonyl, alkoxycarbonylamino, alkylsulfonylamino, dialkylamino, dialkylaminocarbonyl, or dialkylaminosulfonyl having in each case 1 to 5 carbon atoms in the alkyl groups; represent optionally fluorine-, chlorine-, or bromine-substituted alkenyl, alkenyloxy, alkenyloxycarbonyl, alkynyl, alkynyloxy, or alkynyloxycarbonyl having in each case 2 to 5 carbon atoms in the alkenyl or alkynyl groups; represent cycloalkyl, cycloalkenyl, or cycloalkylalkyl having in each case 3 to 6 carbon atoms in the cycloalkyl or cycloalkenyl group and, if appropriate, 1 to 3 carbon atoms in the alkyl moiety; or represent phenyl, phenoxy, phenylthio, pyridyl, furyl, or thienyl, each of which is optionally substituted by nitro, cyano, carboxyl, carbamoyl, thiocarbamoyl, or halogen, by C₁-C₄-alkyl, C₁-C₄-haloalkyl, C₁-C₄-alkoxy, C₁-C₄-haloalkoxy, C₁-C₄-alkylthio, C₁-C₄-haloalkylthio, or C₁-C₄-alkoxy-carbonyl, or
X, Y, and Z together represent a benzo grouping or represent alkylene or alkenylene having in each case up to 4 carbon atoms, where the carbon chain may be interrupted by 1 to 3 nitrogen atoms or 1 to 2 not directly adjacent oxygen atoms, wherein the ring thus formed is optionally substituted by fluorine, chlorine, bromine or alkyl having 1 to 3 carbon atoms, and at least one of the radicals X, Y, or Z represents a saturated or unsaturated, monocyclic or bicyclic heterocyclyl grouping having 4, 6, 9, or 10 carbon atoms and up to 4 nitrogen atoms and/or one oxygen or sulfur atom and optionally additionally containing one or two oxo groupings (C═O), a thioxo grouping (C═S), a —SO grouping, or a —SO₂ grouping and that optionally contains up to 3 substituents selected from the group consisting of nitro; amino; cyano; carboxyl; carbamoyl; thiocarbamoyl; hydroxyl; halogen; optionally cyano-, fluorine-, chlorine-, bromine-, or C₁-C₄-alkoxy-substituted alkyl, alkyl-carbonyl, alkoxy, alkoxy-carbonyl, alkylthio, alkylsulfinyl, alkylsulfonyl, alkylsulfonyloxy, alkylamino, alkylaminocarbonyl, alkoxycarbonylamino, alkylsulfonylamino, dialkylamino, dialkylaminocarbonyl, or dialkylaminosulfonyl having in each case 1 to 5 carbon atoms in the alkyl groups; optionally halogen-substituted alkenyl, alkenyloxy, alkenyloxycarbonyl, alkynyl, alkynyloxy, or alkynyloxycarbonyl having in each case 2 to 5 carbon atoms in the alkenyl or alkynyl groups; cycloalkyl, cycloalkenyl, or cycloalkylalkyl having in each case 3 to 6 carbon atoms in the cycloalkyl or cycloalkenyl group and, if appropriate, 1 to 3 carbon atoms in the alkyl moiety; and phenyl, benzyl, phenoxy, phenylthio, pyridyl, furyl, or thienyl, each of which is optionally substituted by nitro, cyano, carboxyl, carbamoyl, thiocarbamoyl, halogen, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkoxy-carbonyl, methylenedioxy, or difluoromethylenedioxy.

15. A compound of formula (I) according to claim 11 in which
n represents 0, and
X, Y, and Z independently of one another represent hydrogen nitro, cyano, carboxyl, carbamoyl, thiocarbamoyl, hydroxyl, cyanato, thiocyanato, fluorine, chlorine, or bromine; represent optionally cyano-, fluorine-, chlorine-, bromine-, methoxy-, ethoxy-, or n- or i-propoxy-substituted methyl, ethyl, n- or i-propyl, n-, i-, s-, or t-butyl, acetyl, propionyl, n- or i-butyroyl, methoxy, ethoxy, n- or i-propoxy, n-, i-, s-, or t-butoxy, methoxycarbonyl, ethoxycarbonyl, n- or i-propoxycarbonyl, n-, i-, s- or t-butoxycarbonyl, methylthio, ethylthio, n- or i-propylthio, n-, i-, s- or t-butylthio, methylsulfinyl, ethylsulfinyl, n- or i-propylsulfinyl, methylsulfonyl, ethylsulfonyl, n- or i-propylsulfonyl, methylsulfonyloxy, ethylsulfonyloxy, n- or i-propylsulfonyloxy, methylamino, ethylamino, n- or i-propylamino, n-, i-, s-, or t-butylamino, methylaminocarbonyl, ethylaminocarbonyl, n- or i-propylaminocarbonyl, n-, i-, s-, or t-butylaminocarbonyl, methoxycarbonylamino, ethoxycarbonylamino, n- or i-propoxycarbonylamino, n-, i-, s-, or t-butoxycarbonylamino, methylsulfonylamino, ethylsulfonylamino, n- or i-propylsulfonylamino, n-, i-, s-, or t-butylsulfonylamino, dimethylamino, diethylamino, di-n-propyl-amino, di-i-propyl-amino, dimethylaminocarbonyl, diethylaminocarbonyl, dimethylaminosulfonyl, or diethylaminosulfonyl; represent optionally fluorine-, chlorine-, or bromine-substituted ethenyl, propenyl, butenyl, propenyloxy, butenyloxy, propenyloxycarbonyl, butenyloxycarbonyl, ethynyl, propynyl, butynyl, propynyloxy, butynyloxy, propynyloxycarbonyl, or butynyloxycarbonyl; represent cyclopropyl, cyclobutyl, cyclopentyl, cyclohexyl, cyclopentenyl, cyclohexenyl, cyclopropylmethyl, cyclobutylmethyl, cyclopentylmethyl, or cyclohexylmethyl; or represent phenyl, phenoxy, phenylthio, pyridyl, furyl, or thienyl, each of which is optionally substituted by nitro, cyano, carboxyl, carbamoyl, thiocarbamoyl, fluorine, chlorine, bromine, by methyl, ethyl, n- or i-propyl, n-, i-, s-, or t-butyl, difluoromethyl, dichloromethyl, trifluoromethyl, trichloromethyl, chlorodifluoromethyl, fluorodichloromethyl, methoxy, ethoxy, n- or i-propoxy, n-, i-, s-, or t-butoxy, difluoromethoxy, trifluoromethoxy, chlorodifluoromethoxy, methylthio, ethylthio, n- or i-propylthio, n-, i-, s-, or t-butylthio, difluoromethylthio, trifluoromethylthio, chlorodifluoromethylthio, methoxycarbonyl, ethoxycarbonyl, n- or i-propoxycarbonyl, or n-, i-, s-, or t-butoxycarbonyl, or
X, Y, and Z together represent trimethylene, tetramethylene, propene-1,3-diyl, or butadiene1,4-diyl, where the carbon chain may be interrupted by 1 to 3 nitrogen atoms or 1 to 2 not directly adjacent oxygen atoms, wherein the ring thus formed is optionally substituted by fluorine, chlorine, bromine, methyl, or ethyl, and where at least one of the radicals X, Y, or Z represents a saturated or unsaturated, monocyclic or bicyclic heterocyclyl grouping selected from the group consisting of furyl, benzofuryl, tetrahydrofuryl, thienyl, benzothienyl, pyrrolyl, indazolyl, tetrahydroindazolyl, oxopyrrolyl, pyrrolinyl, pyrrolidinyl, 2,5-dioxo-1-azacyclopentyl, pyrazolyl, pyrazolinyl, oxopyrazolinyl, 2-oxo-1,3-diazacyclopentyl, imidazolyl, triazolyl, benzotriazolyl, oxotriazolinyl, tetrazolyl, oxazolyl, benzoxazolyl, thiazolyl, benzothiazolyl, oxadiazolyl, isoxazolyl, thiadiazolyl, pyridinyl, 1,4-dihydro-4-oxopyridin-1-yl, quinolinyl, isoquinolinyl, piperidinyl, oxopiperidinyl, pyrazinyl, pyridazinyl, pyrimidinyl, 2-oxo-1,3-diazacyclohexyl, and morpholinyl, each of which optionally contains up to 3 substituents selected from the group consisting of nitro; amino; cyano; carboxyl; carbamoyl; thiocarbamoyl; hydroxyl; fluorine; chlorine; bromine; iodine; optionally cyano-, fluorine-, chlorine-, bromine-, methoxy-, ethoxy-, or n- or i-propoxy-substituted methyl, ethyl, n- or i-propyl, n-, i-, s-, or t-butyl, acetyl, propionyl, n- or i-butyroyl, methoxy, ethoxy, n- or i-propoxy, n-, i-, s-, or t-butoxy, methoxycarbonyl, ethoxycarbonyl, n- or i-propoxycarbonyl, n-, i-, s-, or t-butoxycarbonyl, methylthio, ethylthio, n- or i-propylthio, n-, i-, s-, or t-butylthio, methylsulfinyl, ethylsulfinyl, n- or i-propylsulfinyl, methylsulfonyl, ethylsulfonyl, n- or i-propylsulfonyl, methylsulfonyloxy, ethylsulfonyloxy, n- or i-propylsulfonyloxy, methylamino, ethylamino, n- or i-propylamino, n-, i-, s-, or t-butylamino, methylaminocarbonyl, ethylaminocarbonyl, n- or i-propylaminocarbonyl, n-, i-, s-, or t-butylaminocarbonyl, methoxycarbonylamino, ethoxycarbonylamino, n- or i-propoxycarbonylamino, n-, i-, s-, or t-butoxycarbonylamino, methylsulfonylamino, ethylsulfonylamino, n- or i-propylsulfonylamino, n-, i-, s-, or t-butylsulfonylamino, dimethylamino, diethylamino, di-n-propylamino, di-i-propyl-amino, dimethylaminocarbonyl, diethylaminocarbonyl, dimethylaminosulfonyl, or diethylaminosulfonyl; optionally fluorine-, chlorine-, or bromine-substituted ethenyl, propenyl, butenyl, propenyloxy, butenyloxy, propenyloxycarbonyl, butenyloxycarbonyl, ethynyl, propynyl, butynyl, propynyloxy, butynyloxy, propynyloxycarbonyl, or butynyloxycarbonyl; cyclopropyl, cyclobutyl, cyclopentyl, cyclohexyl, cyclopentenyl, cyclohexenyl, cyclopropylmethyl, cyclobutylmethyl, cyclopentylmethyl, or cyclohexylmethyl; and phenyl, benzyl, phenoxy, phenylthio, pyridyl, furyl, and thienyl, each of which is optionally substituted by nitro, cyano, carboxyl, carbamoyl, thiocarbamoyl, fluorine, chlorine, bromine, methyl, ethyl, n- or i-propyl, n-, i-, s-, or t-butyl, difluoromethyl, dichloromethyl, trifluoromethyl, trichloromethyl, chlorodifluoromethyl, fluorodichloromethyl, methoxy, ethoxy, n- or i-propoxy, n-, i-, s-, or t-butoxy, difluoromethoxy, trifluoromethoxy, chlorodifluoromethoxy, methylthio, ethylthio, n- or i-propylthio, n-, i-, s-, or t-butylthio, difluoromethylthio, trifluoromethylthio, chlorodifluoromethylthio, methoxycarbonyl, ethoxycarbonyl, n- or i-propoxycarbonyl, n-, i-, s- or t-butoxycarbonyl, methylenedioxy, or difluoromethylenedioxy.

16. A compound of formula (I) according to claim 11 in which
n, R, X, Y, and Z have the meanings given for formula (I) in claim 11, wherein
at least one of the radicals X or Z represents a saturated or unsaturated, monocyclic or bicyclic heterocyclyl grouping selected from the group consisting of furyl, benzofuryl, tetrahydrofuryl, thienyl, benzothienyl, pyrrolyl, indazolyl, tetrahydroindazolyl, oxopyrrolyl, pyrrolinyl, pyrrolidinyl, 2,5-dioxo1-azacyclopentyl, pyrazolyl, pyrazolinyl, oxopyrazolinyl, 2-oxo-1,3-diazacyclopentyl, imidazolyl, triazolyl, benzotriazolyl, oxotriazolinyl, tetrazolyl, oxazolyl, benzoxazolyl, thiazolyl, benzothiazolyl, oxadiazolyl, isoxazolyl, thiadiazolyl, pyridinyl, 1,4-dihydro-4-oxopyridin-1-yl, quinolinyl, isoquinolinyl, piperidinyl, oxopiperidinyl, pyrazinyl, pyridazinyl, pyrimidinyl, 2-oxo-1,3-diazacyclohexyl, and morpholinyl, each of which optionally contains up to 3 substituents selected from the group consisting of nitro; cyano; carboxyl; carbamoyl; thiocarbamoyl; hydroxyl; fluorine; chlorine; bromine; iodine; optionally cyano-, fluorine-, chlorine-, bromine-, methoxy-, ethoxy-, or n- or i-propoxy-substituted methyl, ethyl, n- or i-propyl, n-, i-, s-, or t-butyl, acetyl, propionyl, n- or i-butyroyl, methoxy, ethoxy, n- or i-propoxy, n-, i-, s-, or t-butoxy, methoxycarbonyl, ethoxycarbonyl, n- or i-propoxycarbonyl, n-, i-, s-, or t-butoxycarbonyl, methylthio, ethylthio, n- or i-propylthio, n-, i-, s-, or t-butylthio, methylsulfinyl, ethylsulfinyl, n- or i-propylsulfinyl, methylsulfonyl, ethylsulfonyl, n- or i-propylsulfonyl, methylsulfonyloxy, ethylsulfonyloxy, n- or i-propylsulfonyloxy, methylamino, ethylamino, n- or i-propylamino, n-, i-, s-, or t-butylamino, methylaminocarbonyl, ethylaminocarbonyl, n- or i-propylaminocarbonyl, n-, i-, s-, or t-butylaminocarbonyl, methoxycarbonylamino, ethoxycarbonylamino, n- or i-propoxycarbonylamino, n-, i-, s-, or t-butoxycarbonylamino, methylsulfonylamino, ethylsulfonylamino, n- or i-propylsulfonylamino, n-, i-, s-, or t-butylsulfonylamino, dimethylamino, diethylamino, di-n-propyl-amino, di-i-propylamino, dimethylaminocarbonyl, diethylaminocarbonyl, dimethylaminosulfonyl, or diethylaminosulfonyl; optionally fluorine-, chlorine-, or bromine-substituted ethenyl, propenyl, butenyl, propenyloxy, butenyloxy, propenyloxycarbonyl, butenyloxycarbonyl, ethynyl, propynyl, butynyl, propynyloxy, butynyloxy, propynyloxycarbonyl, or butynyloxycarbonyl; cyclopropyl, cyclobutyl, cyclopentyl, cyclohexyl, cyclopentenyl, cyclohexenyl, cyclopropylmethyl, cyclobutylmethyl, cyclopentylmethyl, or cyclohexylmethyl; and phenyl, benzyl, phenoxy, phenylthio, pyridyl, furyl, or thienyl, each of which is optionally substituted by nitro, cyano, carboxyl, carbamoyl, thiocarbamoyl, fluorine, chlorine, bromine, methyl, ethyl, n- or i-propyl, n-, i-, s-, or t-butyl, difluoromethyl, dichloromethyl, trifluoromethyl, trichloromethyl, chlorodifluoromethyl, fluorodichloromethyl, methoxy, ethoxy, n- or i-propoxy, n-, i-, s-, or t-butoxy, difluoromethoxy, trifluoromethoxy, chlorodifluoromethoxy, methylthio, ethylthio, n- or i-propylthio, n-, i-, s-, or t-butylthio, difluoromethylthio, trifluoromethylthio, chlorodifluoromethylthio, methoxycarbonyl, ethoxycarbonyl, n- or i-propoxycarbonyl, or n-, i-, s-, or t-butoxycarbonyl, and
the other of the radicals X or Z represents hydrogen.

17. A compound of formula (I) according to claim 11 in which
n, R, X and Z have the meanings given for formula (I) in claim 11, wherein
X and Z represent hydrogen, and
Y represents a saturated or unsaturated, monocyclic or bicyclic heterocyclyl grouping selected from the group consisting of furyl, benzofuryl, tetrahydrofuryl, thienyl, benzothienyl, pyrrolyl, oxopyrrolyl, pyrrolinyl, pyrrolidinyl, pyrazolyl, pyrazolinyl, oxopyrazolinyl, 2-oxo-1,3-diazacyclopentyl, triazolyl, benzotriazolyl, oxotriazolinyl, tetrazolyl, oxazolyl, benzoxazolyl, thiazolyl, benzothiazolyl, oxadiazolyl, isoxazolyl, thiadiazolyl, pyridinyl, quinolinyl, isoquinolinyl, piperidinyl, oxopiperidinyl, pyrazinyl, pyridazinyl, pyrimidinyl, 2-oxo-1,3-diazacyclohexyl, and morpholinyl, each of which optionally contains up to 3 substituents selected from the group consisting of nitro; cyano; carboxyl; carbamoyl; thiocarbamoyl; hydroxyl; fluorine; chlorine; bromine; iodine; optionally cyano-, fluorine-, chlorine-, bromine-, methoxy-, ethoxy-, or n- or i-propoxy-substituted methyl, ethyl, n- or i-propyl, n-, i-, s-, or t-butyl, acetyl, propionyl, n- or i-butyroyl, methoxy, ethoxy, n- or i-propoxy, n-, i-, s-, or t-butoxy, methoxycarbonyl, ethoxycarbonyl, n- or i-propoxycarbonyl, n-, i-, s-, or t-butoxycarbonyl, methylthio, ethylthio, n- or i-propylthio, n-, i-, s-, or t-butylthio, methylsulfinyl, ethylsulfinyl, n- or i-propylsulfinyl, methylsulfonyl, ethylsulfonyl, n- or i-propylsulfonyl, methylsulfonyloxy, ethylsulfonyloxy, n- or i-propylsulfonyloxy, methylamino, ethylamino, n- or i-propylamino, n-, i-, s-, or t-butylamino, methylaminocarbonyl, ethylaminocarbonyl, n- or i-propylaminocarbonyl, n-, i-, s-, or t-butylaminocarbonyl, methoxycarbonylamino, ethoxycarbonylamino, n- or i-propoxycarbonylamino, n-, i-, s-, or t-butoxycarbonylamino, methylsulfonylamino, ethylsulfonylamino, n- or i-propylsulfonylamino, n-, i-, s-, or t-butylsulfonylamino, dimethylamino, diethylamino, di-n-propylamino, di-i-propyl-amino, dimethylaminocarbonyl, diethylaminocarbonyl, dimethylaminosulfonyl, or diethylaminosulfonyl; optionally fluorine-, chlorine-, or bromine-substituted ethenyl, propenyl, butenyl, propenyloxy, butenyloxy, propenyloxycarbonyl, butenyloxycarbonyl, ethynyl, propynyl, butynyl, propynyloxy, butynyloxy, propynyloxycarbonyl, or butynyloxycarbonyl; cyclopropyl, cyclobutyl, cyclopentyl, cyclohexyl, cyclopentenyl, cyclohexenyl, cyclopropylmethyl, cyclobutylmethyl, cyclopentylmethyl, or cyclohexylmethyl; and phenyl, benzyl, phenoxy, phenylthio, pyridyl, furyl, or thienyl, each of which is optionally substituted by nitro, cyano, carboxyl, carbamoyl, thiocarbamoyl, fluorine, chlorine, bromine, by methyl, ethyl, n- or i-propyl, n-, i-, s-, or t-butyl, difluoromethyl, dichloromethyl, trifluoromethyl, trichloromethyl, chlorodifluoromethyl, fluorodichloromethyl, methoxy, ethoxy, n- or i-propoxy, n-, i-, s-, or t-butoxy, difluoromethoxy, trifluoromethoxy, chlorodifluoromethoxy, methylthio, ethylthio, n- or i-propylthio, n-, i-, s-, or t-butylthio, difluoromethylthio, trifluoromethylthio, chlorodifluoromethylthio, methoxycarbonyl, ethoxycarbonyl, n- or i-propoxycarbonyl, or n-, i-, s-, or t-butoxycarbonyl.

18. A pesticide comprising one or more compounds of formula (I) as claimed in claim 11 and one or more additives an/or surfactants.

19. A method for controlling pests comprising allowing an effective amount of one or more compounds of formula (I) as claimed in claim 11 to act on pests and/or their habitat.

20. A method for controlling pests comprising allowing an effective amount of a composition as claimed in claim 18 to act on pests and/or their habitat.

21. A process for preparing pesticides comprising mixing one or more compounds of formula (I) as claimed in claim 11 with one or more extenders and/or surfactants.
